(19) United States
(12) Patent Application Publication
(10) Pub. No.: US 2016/0168119 A1
(43) Pub. Date: Jun. 16, 2016

(54) AUTOTAXIN INHIBITORS

(71) Applicants: Urs BAETTIG, Reinach (CH); David BEATTIE, Horsham (GB); Darren Mark LEGRAND, Horsham (GB); Andrew Stuart LISTER, Broadbridge Heath (GB); Jeffrey MCKENNA, Carlisle, MA (US); David William PEARCE, Horsham (GB); David Andrew SANDHAM, Horsham (GB); Emily STANLEY, Horsham (GB); Oliver Ross STEWARD, Horsham (GB); Christopher THOMSON, Belmont, MA (US)

(72) Inventors: Urs BAETTIG, Reinach (CH); David BEATTIE, Horsham (GB); Darren Mark LEGRAND, Horsham (GB); Andrew Stuart LISTER, Broadbridge Heath (GB); Jeffrey MCKENNA, Carlisle, MA (US); David William PEARCE, Horsham (GB); David Andrew SANDHAM, Horsham (GB); Emily STANLEY, Horsham (GB); Oliver Ross STEWARD, Horsham (GB); Christopher THOMSON, Belmont, MA (US)

(73) Assignee: Novartis AG, Basel (CH)

(21) Appl. No.: 14/906,187

(22) PCT Filed: Jul. 16, 2014
(86) PCT No.: PCT/IB2014/063142

§ 371 Date: Jan. 19, 2016

(30) Foreign Application Priority Data
Jul. 18, 2013 (EP) 13177062.0

(51) Int. Cl. C07D 401/12; C07D 403/12; C07D 257/04; C07D 405/12; C07D 417/12; C07D 413/12

(52) U.S. Cl. CPC C07D 401/12; C07D 417/12; C07D 413/12; C07D 257/04; C07D 405/12; C07D 403/12

(57) ABSTRACT

The present invention relates to novel compounds that are autotaxin inhibitors, processes for their preparation, pharmaceutical compositions and medicaments containing them and to their use in diseases and disorders mediated by autotaxin.

### TECHNICAL FIELD

The present invention relates to novel compounds that are autotaxin inhibitors, processes for their preparation, pharmaceutical compositions and medicaments containing them and to their use in diseases and disorders mediated by autotaxin.

### BACKGROUND

Autotaxin (ATX), also known as ectonucleotide pyrophosphatase/phosphodiesterase (ENPP2), is a secreted ectoenzyme known to possess lysophospholipase D activity (Umezu-Goto et al., 2002), and is responsible for producing the bioactive lipid mediator lysophosphatidic acid (LPA) by the hydrolysis of lysophosphatidylcholine (LPC) (Tokumura et al., 2002). LPA is highly implicated in the pathogenesis of a number of physio-pathological diseases, including cancer (Liu et al., 2009; Mills & Moolenaar, 2003), neuropathic pain (Inoue et al., 2004) and fibrosis (Tager et al., 2008). Following the production of LPA, the lipid binds to specific G protein-coupled receptors of which there are seven known isoforms (Noguchi et al., 2009). Binding of LPA activates multiple signalling pathways (Mills & Moolenaar, 2003) including cell migration (van Dijk et al., 1998), proliferation and survival (Brindley, 2004). Other cellular responses include smooth muscle contraction, apoptosis and platelet aggregation (Tigyi & Parrill, 2003).

ATX was originally identified as a cell motility-stimulating factor following isolation from human A2058 melanoma cells (Stracke et al., 1992). Subsequent work on the enzyme was focused towards its role as a motility factor due to its aberrant expression in many cancer types including breast and renal cancer (Stassar et al., 2001), Hodgkin's lymphoma (Baumforth et al., 2005), follicular lymphoma (Masuda et al., 2008), as well as fibrosis of the lung and kidney (Hama et al., 2004). Ten years following its discovery, ATX was characterised as a secreted lysophospholipase (lysoPLD) (Tokumura et al., 2002; Gesta et al., 2002). Since then ATX gene knockout mice have shown that the ATX-LPA signalling axis plays a vital role during embryonic development of the cardiovascular and neural system (Tanaka et al., 2006; van Meeteren et al., 2006), resulting in early embryonic lethality (Bachner et al., 1999).

ATX belongs to a family of proteins called nucleotide pyrophosphatase/phosphodiesterase (NPP), encoded for by the gene ENPP. The family consists of seven structurally related enzymes (ENPP 1-7) conserved within vertebrates which are numbered according to their discovery. They were originally defined by their ability to hydrolyse pyrophosphate or phosphodiester bonds of various nucleotides and nucleotides derivatives in vitro (Stefan et al., 1999; Goding et al., 1998; Gijsbers et al., 2001), though ENPP2 and choline phosphate esters (ENPP6 & 7) have specific activity for other extracellular non-nucleotide molecules. ENPP2 (ATX) is unique within the family as it is the only secreted protein, whereas other ENPP members are transmembrane proteins (Stefan et al., 2005).

Hence, there is a need for potent inhibitors of ATX.

### SUMMARY OF THE INVENTION

In a first aspect, the invention relates to a compound according to formula (I)

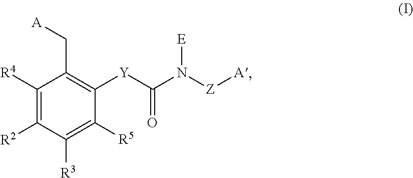

or a pharmaceutically acceptable salt thereof,
wherein
A is selected from the group consisting of

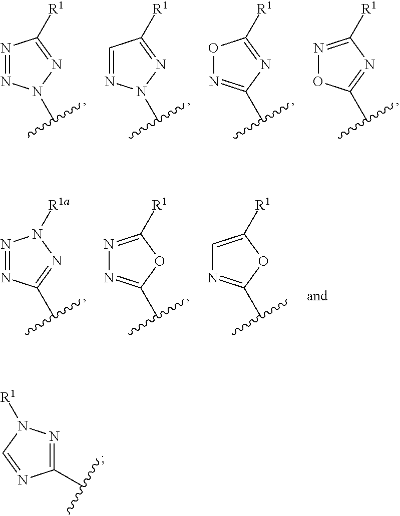

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heteroaryl which heteroaryl is unsubstituted or substituted with 1 to 3 substituents independently selected from the group consisting of X;
(ii) phenyl which phenyl is unsubstituted or substituted with 1 to 3 substituents independently selected from the group consisting of X;
(iii) 5 to 10 membered fused bicyclic ring system which 5 to 10 membered fused bicyclic ring system is unsubstituted or substituted with 1 to 3 substituents independently selected from the group consisting of X;
(iv) 5 or 6 membered heterocyclyl which heterocyclyl is unsubstituted or substituted with 1 to 3 substituents independently selected from the group consisting of X;
(v) C₁₋₄ alkyl;
(vi) C₁₋₄ alkoxy;
(vii) C₁₋₄ alkoxy C₁₋₄ alkyl;
(viii) C₁₋₄ haloalkyl;
(ix) hydroxy C₁₋₄ alkyl;
(x) C₃₋₆ cycloalkyl;

### (xi) —C(═O)R^{Bc};

(xii) —C(═O)OR^{Be};
(xiii) —NR^{Bd}—C(═O)R^{Bc};
(xiv) —NR^{Bd}—C(═O)OR^{Bc};
(xv) —C(═O)NR^{Bc}R^{Bd};
(xvi) —NR^{Bd}R^{Be};
(xvii) —NR^{Bd}—S(O)₂—R^{Bf};
(xviii) —S(O)₂—NR^{Bd}R^{Be};
(xix) —S(O)₂—R^{Bf};
(xx) halogen;
(xxi) OH;
(xxii) oxo; and
(xxiii) CN;
R^{Bc}, R^{Be} and R^{Bf} are independently selected from the group consisting of H, C₁₋₄alkyl, —(CR^{Ba}R^{Bb})ₙ—C₃₋₆cycloalkyl, hydroxyC₁₋₄alkyl, C₁₋₄haloalkyl, C₁₋₄alkoxyC₁₋₄alkyl, OH, —(CR^{Ba}R^{Bb})ₙ-5 or 6 membered heteroaryl, —(CR^{Ba}R^{Bb})ₙ-phenyl and —(CR^{Ba}R^{Bb})ₙ-5 or 6 membered heterocyclyl, wherein the C₃₋₆cycloalkyl, heteroaryl, phenyl and heterocyclyl are unsubstituted or substituted by 1 to 3 substituents selected from the group consisting of X;
R^{Bd} is selected from the group consisting of H and C₁₋₄alkyl; or
R^{Bd} and R^{Be} together with the nitrogen atom to which they are attached form a 5 to 6 membered heterocyclyl which heterocyclyl is unsubstituted or substituted by 1 to 3 substituents selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆ cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, (CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Ba}, R^{Bb}, R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
n and q are independently selected from the group consisting of 0, 1, 2, 3 and 4.

In another aspect, the invention relates to processes for preparing compounds of the first aspect.

In another aspect, the invention relates to the use of compounds of the first aspect in the treatment of a disease or condition selected from fibrosis, pruritus, cirrhosis, cancer, diabetes, kidney diseases and pain.

In a further aspect, the invention relates to pharmaceutical compositions and combinations comprising a compound of the first aspect.

### DESCRIPTION OF THE EMBODIMENTS

### Embodiment 1

A Compound of formula (I)

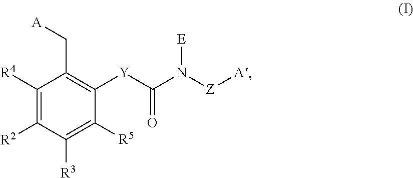

or a pharmaceutically acceptable salt thereof,
wherein
A is selected from the group consisting of

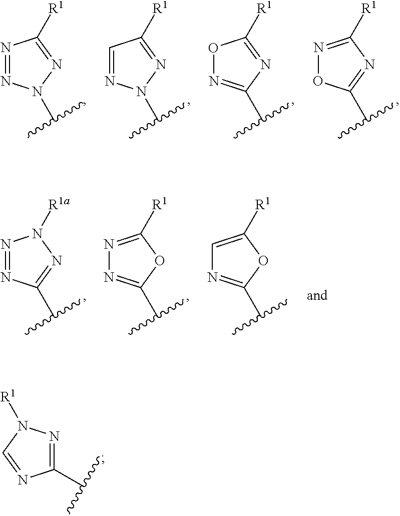

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R⁴ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heteroaryl which heteroaryl is unsubstituted or substituted with 1 to 3 substituents independently selected from the group consisting of X;
(ii) phenyl which phenyl is unsubstituted or substituted with 1 to 3 substituents independently selected from the group consisting of X;
(iii) 5 to 10 membered fused bicyclic ring system which 5 to 10 membered fused bicyclic ring system is unsubstituted or substituted with 1 to 3 substituents independently selected from the group consisting of X;
(iv) 5 or 6 membered heterocyclyl which heterocyclyl is unsubstituted or substituted with 1 to 3 substituents independently selected from the group consisting of X;
(v) C₁₋₄ alkyl;
(vi) C₁₋₄ alkoxy;
(vii) C₁₋₄ alkoxy C₁₋₄ alkyl;
(viii) C₁₋₄ haloalkyl;
(ix) hydroxy C₁₋₄ alkyl;
(x) C₃₋₆ cycloalkyl;

### (xi) —C(═O)R^{Bc};

(xii) —C(═O)OR^{Be};
(xiii) —NR^{Bd}—C(═O)R^{Bc};
(xiv) —NR^{Bd}—C(═O)OR^{Bc};
(xv) —C(═O)NR^{Bc}R^{Bd};
(xvi) —NR^{Bd}R^{Be};
(xvii) —NR^{Bd}—S(O)₂—R^{Bf};
(xviii) —S(O)₂—NR^{Bd}R^{Be};
(xix) —S(O)₂—R^{Bf};
(xx) halogen;
(xxi) OH;
(xxii) oxo; and
(xxiii) CN;
R^{Bc}, R^{Be} and R^{Bf} are independently selected from the group consisting of H, C₁₋₄alkyl, —(CR^{Ba}R^{Bb})ₙ—C₃₋₆cycloalkyl, hydroxyC₁₋₄alkyl, C₁₋₄haloalkyl, C₁₋₄alkoxyC₁₋₄alkyl, OH, —(CR^{Ba}R^{Bb})ₙ-5 or 6 membered heteroaryl, —(CR^{Ba}R^{Bb})ₙ-phenyl and —(CR^{Ba}R^{Bb})ₙ-5 or 6 membered heterocyclyl, wherein the C₃₋₆cycloalkyl, heteroaryl, phenyl and heterocyclyl are unsubstituted or substituted by 1 to 3 substituents selected from the group consisting of X;
R^{Bd} is selected from the group consisting of H and C₁₋₄alkyl; or
R^{Bd} and R^{Be} together with the nitrogen atom to which they are attached form a 5 to 6 membered heterocyclyl which heterocyclyl is unsubstituted or substituted by 1 to 3 substituents selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆ cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Ba}, R^{Bb}, R^{Xa} and R^{Xb} are independently selected from the group consisting of H, OH, C₁₋₄ alkyl and C₁₋₄ alkoxy;

R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or

R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
n and q are independently selected from the group consisting of 0, 1, 2, 3 and 4.

### Embodiment 2

A compound or salt according to embodiment 1, of formula (I)

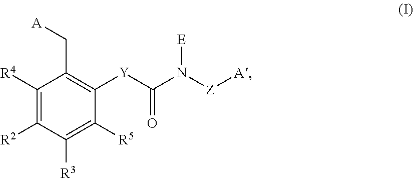

or a pharmaceutically acceptable salt thereof,
wherein
A is selected from the group consisting of

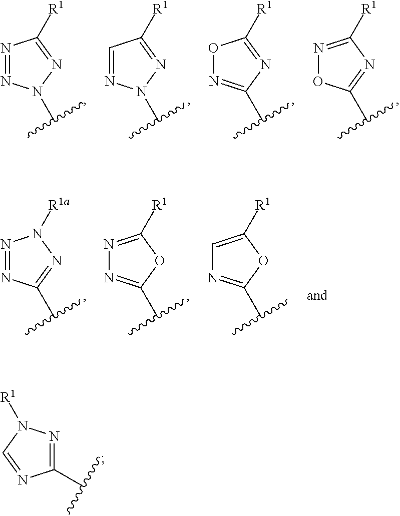

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heteroaryl which heteroaryl is unsubstituted or substituted with 1 to 3 substituents independently selected from the group consisting of X;
(ii) phenyl which phenyl is unsubstituted or substituted with 1 to 3 substituents independently selected from the group consisting of X;
(iii) 5 to 10 membered fused bicyclic ring system which is unsubstituted or substituted with 1 to 3 substituents independently selected from the group consisting of X;
(iv) 5 or 6 membered heterocyclyl which heterocyclyl is unsubstituted or substituted with 1 to 3 substituents independently selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
q is selected from the group consisting of 0, 1 and 2.

### Embodiment 3

A compound or salt according to embodiment 1, of formula (I)

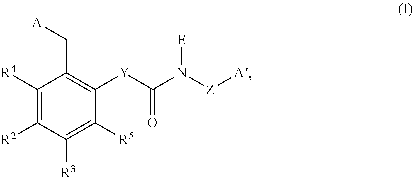

or a pharmaceutically acceptable salt thereof,
wherein
A is selected from the group consisting of

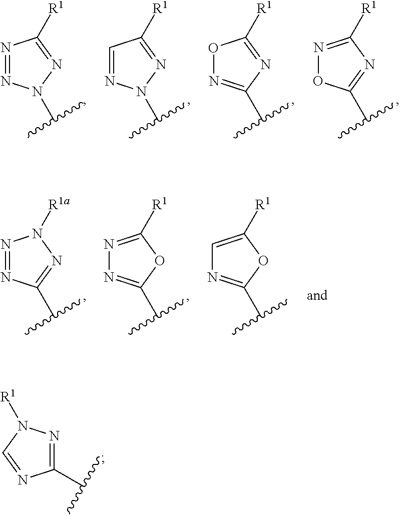

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heteroaryl which heteroaryl is unsubstituted or substituted with 1 to 3 substituents independently selected from the group consisting of X;
(ii) 5 or 6 membered heterocyclyl which heterocyclyl is unsubstituted or substituted with 1 to 3 substituents independently selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, (CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
q is selected from the group consisting of 0, 1 and 2.

### Embodiment 4

A compound or salt according to embodiment 1, of formula (I)

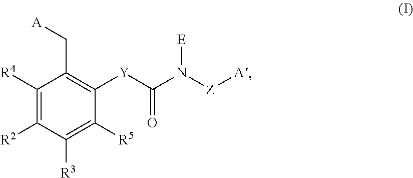

or a pharmaceutically acceptable salt thereof,
wherein
A is selected from the group consisting of

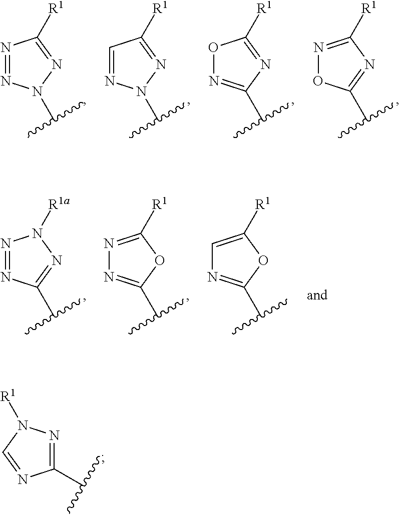

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁-6 alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heterocyclyl which heterocyclyl is unsubstituted or substituted with 1 to 3 substituents independently selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆ cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, (CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
q is selected from the group consisting of 0, 1 and 2.

### Embodiment 5

A compound or salt according to embodiment 1, of formula (I)

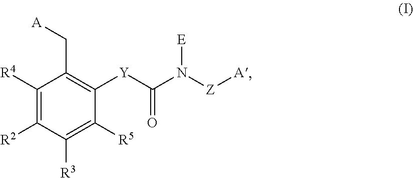

or a pharmaceutically acceptable salt thereof,
wherein
A is selected from the group consisting of

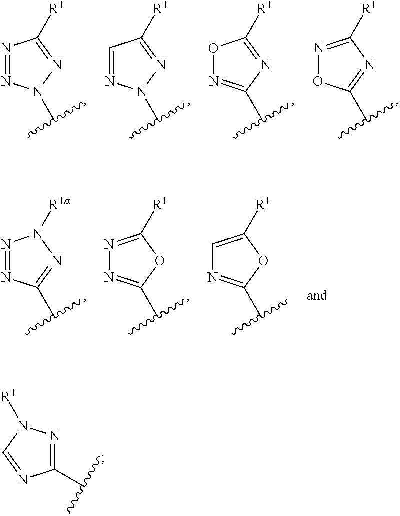

R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁-6 alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) -piperidin-4-yl;
(ii) -pyrrolidin-3-yl;
(iii) -oxadiazol-5-yl;
(iv) -thiazol-2-yl;
(v) -pyrazol-5-yl;
(vi) -pyrazol-3-yl;
(vii) -isoxazol-5-yl;
(viii) -tetrahydrofuran-2-yl
each of (i) to (vii) is unsubstituted or substituted with 1 to 3 substituents independently selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
q is selected from the group consisting of 0, 1 and 2.

### Embodiment 6

A compound or salt according to any one of embodiments 1 to 5, wherein R² is halogen, —CF₃, —CF₂H, —OCF₃, —OCF₂H, —OCH₃, —CH₃ or CN, and R³, R⁴ and R⁵ are H.

### Embodiment 7

A compound or salt according to any one of embodiments 1 to 6, wherein A is selected from the group consisting of

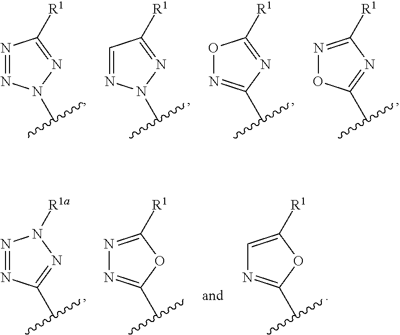

### Embodiment 8

A compound or salt according to any one of embodiments 1 to 7, wherein A is selected from the group consisting of

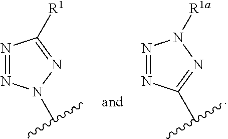

### Embodiment 9

A compound or salt according to any one of embodiments 1 to 8, wherein A is

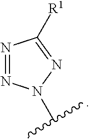

### Embodiment 10

A compound or salt according to any one of embodiments 1 to 9, wherein R¹ is methyl.

### Embodiment 11

A compound or salt according to any one of embodiments 1 to 10, wherein R² is —CF₃, chloro, —CF₂H or —OCF₃.

### Embodiment 12

A compound or salt according to embodiment 11, wherein R² is —CF₃ or chloro.

### Embodiment 13

A compound or salt according to any one of embodiments 1 to 12, wherein Y is selected from the group consisting of —CH═CH—, —CH₂—CH₂— and —C(CH₃)═CH—, particularly —CH═CH—.

### Embodiment 13.1

A compound or salt according to any one of embodiments 1 to 12, wherein Y is selected from the group consisting of —O—CH₂—, —CH₂—O— and —C(CH₃)═CH—, more particularly —O—CH₂— and —C(CH₃)═CH—.

### Embodiment 14

A compound or salt according to any one of embodiments 1 to 13, wherein Z is selected from the group consisting of —(CR^{7a}R^{7b})ₘ—, wherein R^{7a} and R^{7b} are H and m is 0 or 1.

### Embodiment 15

A compound according to embodiment 1 selected from the group consisting of
(E)-tert-butyl 4-((3-(4-chloro-2-((5-methyl-2H-tetrazol-2-yl)methyl)phenyl)-N-methylacrylamido)methyl)piperidine-1-carboxylate;
(E)-tert-butyl 4-(N-ethyl-3-(2-((5-methyl-2H-tetrazol-2-yl)methyl)-4-(trifluoromethyl)phenyl)acrylamido)piperidine-1-carboxylate;
(E)-3-(4-chloro-2-((5-methyl-2H-tetrazol-2-yl)methyl)phenyl)-N-ethyl-N-(pyridin-4-ylmethyl)acrylamide;
(E)-3-(4-chloro-2-((5-methyl-2H-tetrazol-2-yl)methyl)phenyl)-N-methyl-N-((4-methylthiazol-2-yl)methyl)acrylamide;
(E)-3-(4-chloro-2-((5-methyl-2H-tetrazol-2-yl)methyl)phenyl)-N-methyl-N-((3-methylisoxazol-5-yl)methyl)acrylamide;
(E)-3-(4-chloro-2-((5-methyl-2H-tetrazol-2-yl)methyl)phenyl)-N-ethyl-N-(2-methoxyethyl)acrylamide;
(E)-3-(4-chloro-2-((5-methyl-2H-tetrazol-2-yl)methyl)phenyl)-N-ethyl-N-(2-hydroxy-2-(3-hydroxyphenyl)ethyl)acrylamide;
(E)-N-((1-tert-butyl-5-oxopyrrolidin-3-yl)methyl)-3-(4-chloro-2-((5-methyl-2H-tetrazol-2-yl)methyl)phenyl)-N-methylacrylamide;
(E)-3-(4-chloro-2-((5-methyl-2H-tetrazol-2-yl)methyl)phenyl)-N-methyl-N-((tetrahydrofuran-2-yl)methyl)acrylamide;
(E)-N-((1-(4-fluorobenzoyl)piperidin-4-yl)methyl)-N-methyl-3-(2-((5-methyl-2H-tetrazol-2-yl)methyl)-4-(trifluoromethyl)phenyl)acrylamide;
(E)-N-((1-(cyclopropylsulfonyl)piperidin-4-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-((1-tosylpiperidin-4-yl)methyl)acrylamide;
(E)-N-((1-(4-fluorobenzyl)piperidin-4-yl)methyl)-N-methyl-3-(2-((5-methyl-2H-tetrazol-2-yl)methyl)-4-(trifluoromethyl)phenyl)acrylamide;
(E)-N-((1,3-dimethyl-1H-pyrazol-5-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-(2-phenoxyethyl)acrylamide;
(E)-N-methyl-3-(2-((5-methyl-2H-tetrazol-2-yl)methyl)-4-(trifluoromethyl)phenyl)-N-((4-methylthiazol-2-yl)methyl)acrylamide;
(E)-N-methyl-3-(2-((5-methyl-2H-tetrazol-2-yl)methyl)-4-(trifluoromethyl)phenyl)-N-((5-phenyl-1,3,4-oxadiazol-2-yl)methyl)acrylamide;
(E)-N-(2-(4-methoxybenzylamino)-2-oxoethyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-(4-(pyridin-3-yl)butyl)acrylamide;
(E)-N-methyl-3-(2-((5-methyl-2H-tetrazol-2-yl)methyl)-4-(trifluoromethyl)phenyl)-N-((4-phenylthiazol-2-yl)methyl)acrylamide;
(E)-N-((1-tert-butyl-5-oxopyrrolidin-3-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-((3-phenyl-1,2,4-oxadiazol-5-yl)methyl)acrylamide; and
(E)-N-(benzo[d]thiazol-2-ylmethyl)-N-methyl-3-(2-((5-methyl-2H-tetrazol-2-yl)methyl)-4-(trifluoromethyl)phenyl)acrylamide;
   or a pharmaceutically acceptable salt thereof.

### DEFINITIONS

“Halo” or “halogen”, as used herein, may be fluoro, chloro, bromo or iodo.

“C₁₋₄ alkyl”, as used herein, denotes straight chain or branched alkyl having 1-4 carbon atoms. If a different number of carbon atoms is specified, such as C₆ or C₃, then the definition is to be amended accordingly, such as “C₁-C₄ alkyl” will represent methyl, ethyl, propyl, isopropyl, butyl, isobutyl, sec-butyl and tert-butyl.

“C₁₋₄ haloalkyl”, as used herein, denotes straight chain or branched alkyl having 1-4 carbon atoms with at least one hydrogen substituted with a halogen. If a different number of carbon atoms is specified, such as C₆ or C₃, then the definition is to be amended accordingly, such as “C₁-C₄-Haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃, CF₂H—, CF₃CF₂CHCF₃ or CF₃CF₂CF₂CF₂—.

“C₁₋₄ alkoxy”, as used herein, refers to an —O—C₁₋₄ alkyl group wherein C₁₋₄ alkyl is as defined herein. Examples of such groups include methoxy, ethoxy, propoxy, butoxy, pentoxy or hexoxy and the like. As for alkyl unless a particular structure is specified the terms propoxy, butoxy etc include all straight and branched chain forms having the appropriate number of carbon atoms e.g. propoxy includes n-propoxy and isopropoxy.

“C₁₋₄ haloalkoxy” as used herein refers to an —O—C₁₋₄ alkyl group wherein C₁₋₄ alkyl is as defined herein and substituted with one or more halogen groups, e.g. —O—CF₃.

“C₁₋₄ alkoxy C₁₋₄ alkyl” as used herein refers to an —C₁₋₃ alkyl-O—C₁₋₃ alkyl group wherein C₁₋₃ alkyl is as defined herein. Examples of such groups include methoxyethyl, methoxypropyl, ethoxypropyl.

“hydroxyl C₁₋₄alkyl”, as used herein, denotes a straight chain or branched alkyl having 1-4 carbon atoms with at least one hydrogen substituted with a hydroxy group. If a different number of carbon atoms is specified, such as C₆ or C₃, then the definition is to be amended accordingly, such as “C₁-C₄ hydroxyalkyl” will represent methyl, ethyl, propyl, isopropyl, butyl, isobutyl, sec-butyl and tert-butyl that have at least one hydrogen substituted with hydroxy.

“C₃₋₆ cycloalkyl” as used herein refers to a saturated monocyclic hydrocarbon ring of 3 to 6 carbon atoms. Examples of such groups include cyclopropyl, cyclobutyl, cyclopentyl and cyclohexyl. If a different number of carbon atoms is specified, then the definition is to be amended accordingly.

“Oxo” refers to ═O.

The term “5 or 6 membered heteroaryl” refers to a 5 or 6 membered aromatic ring system which contains 1 to 3 heteroatoms selected from oxygen, nitrogen or sulfur. Examples of 5-membered heteroaryl rings in this instance include furanyl, pyrrolyl, oxazolyl, thiazolyl, imidazolyl, oxadiazolyl, thiadiazolyl, triazolyl, isothiazolyl, isoxazolyl, thiophenyl, or pyrazolyl. Examples of 6-membered heteroaryl rings include pyridinyl, pyridazinyl, pyrimidinyl, pyrazinyl, or triazinyl.

The term “5 or 6 membered heterocyclyl ring” refers to a 5 or 6 membered saturated or partially unsaturated ring system which contains 1 to 3 heteroatoms selected from oxygen, nitrogen or sulphur. Suitable examples of such ring systems include pyrrolidinyl, piperidinyl, piperazinyl, morpholinyl, homomorpholinyl, thiomorpholinyl, tetrahydrofuranyl, tetrahydropyranyl, tetrahydrothiophenyl, pyrrolinyl, or oxazolinyl.

The term “9 to 10 membered fused bicyclic ring system” refers to a “5 or 6 membered heteroaryl” or a “5 or 6 membered heterocyclyl ring” as defined hereinbefore wherein two neighbouring atoms (i.e. atoms bonded directly to each other) of the “5 or 6 membered heteroaryl” or “5 or 6 membered heterocyclyl ring” form together a second ring which second ring contains 0, 1 or 2 heteroatoms selected from oxygen, nitrogen and sulphur.

The term “a,” “an,” “the” and similar terms used in the context of the present invention (especially in the context of the claims) are to be construed to cover both the singular and plural unless otherwise indicated herein or clearly contradicted by the context.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, when one embodiment refers to several other embodiments by using the term “according to any one of”, for example “according to any one of embodiments 1 to 5”, then said embodiment refers not only to embodiments indicated by the integers such as 1 and 2 but also to embodiments indicated by numbers with a decimal component such as 1.1, 1.2 or 2.1, 2.2, 2.3. For example, “according to any one of embodiments 1 to 3” means according to any one of embodiments 1, 1.1, 2, 3, 3.1, 3.2, 3.3, 3.4, 3.5, 3.6, 3.7.

Various embodiments of the invention are described herein. It will be recognized that features specified in each embodiment may be combined with other specified features to provide further embodiments.

The term “compounds of the (present) invention” or “a compound of the (present) invention” refers to a compound as defined in any one of embodiments 1 to 6.

The compounds of the present invention may be prepared by the routes described in the following Schemes or the Examples.

In the following general methods, R4, R2, R3, R5, Z, A′ and E are as previously defined in the embodiments, or limited to designations in the schemes. Unless otherwise stated, starting materials are either commercially available or are synthesised by known procedures.

Compounds of formula I, where Y is —CH2-CH2-, may be prepared from compounds of formula I, where Y is —CH═CH—, by hydrogenation over a suitable catalyst, such as palladium on carbon or platinum on carbon, in a suitable solvent, such as ethanol at room temperature.

Compounds of formula I may be prepared from compounds of formula II and III as illustrated in Scheme 1, by using a suitable coupling reagent, such as T3P or HATU and an organic base such as triethylamine, in a suitable organic solvent such as DMF, ethylacetate or dichloromethane, at a temperature from 25° C. to 50° C., preferably at 25° C.

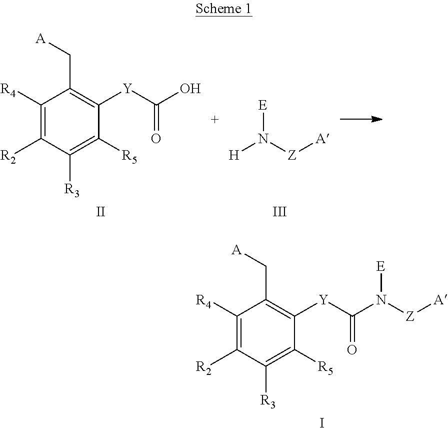

wherein A, E, Y, Z, A′, R², R³, R⁴ and R⁵ are as defined in embodiment 1.

Alternatively, when Y is —CH═CH—, compounds of formula I may be prepared from compounds of formula IV and V as illustrated in Scheme 2, wherein Y1 is a halogen group, preferably a bromine. The coupling reaction requires suitable “Heck” conditions of a palladium catalyst and phosphine ligand, such as palladium II acetate and tri-o-tolyl phosphine, and a suitable organic base, such as triethylamine, in an organic solvent such as DMF, at a temperature of 60° C. to 120° C., preferably at 80° C. Those skilled in the art will appreciate that other catalyst/phosphine combinations and solvents may also be suitable. Compounds of formula V may be prepared by compounds of formula III by reaction with acryloyl chloride in the presence of a base, such as triethylamine, in a suitable solvent such as dichloromethane at a temperature of 0° C.

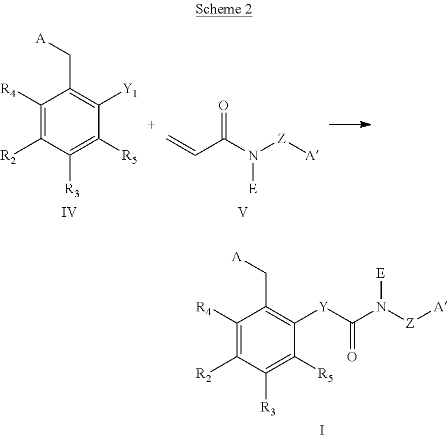

When Y is CH═CH, compounds of formula II can be prepared from compounds of formula IV, where Y1 is a halogen group, preferably bromine, as illustrated in Scheme 3.

Step i) Compounds of formula VI can be prepared from compounds of formula IV using “Heck” coupling conditions with an acrylate, such as ethyl acrylate, and a catalyst such as palladium II acetate and tri-o-tolyl phosphine, and a suitable organic base, such as triethylamine in an organic solvent such as DMF, at a temperature of 60° C. to 120° C., preferably at 80° C. Those skilled in the art will appreciate that other catalyst/phosphine combinations and solvents may also be suitable.

Step ii) Compounds of formula II can be prepared from compounds of formula VI using an alkaline solution, such as sodium hydroxide solution, in an organic solvent such as ethanol, at a temperature of 0° C. to 50° C., preferably at room temperature.

When Y is —CH2-CH2-, compounds of formula II can be prepared from compounds of formula IV as illustrated to Scheme 3 by replacing the ethyl acrylate in step i with acrolein diethylacetal.

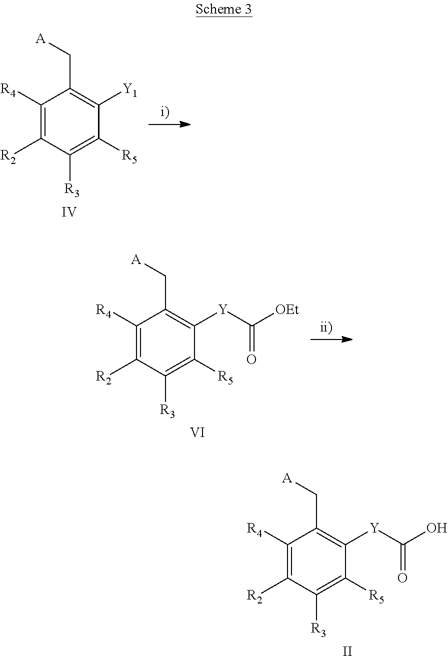

When A is a nitrogen linked tetrazole or triazole, compounds of formula IV can be prepared as illustrated in Scheme 4 from compounds of formula VII, where Y′ is a halogen, preferably bromine or an ester group, preferably methylester, by an alkylation reaction using a suitable base, such as potassium carbonate, in a suitable solvent such as DMF at a temperature of −10° C. to 50° C., preferably 0° C. to room temperature. The heterocycle A is either commercially available or synthesized by known procedures.

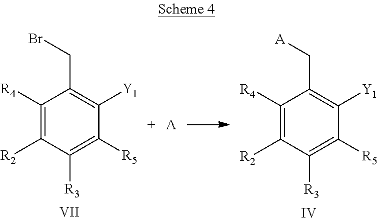

When A is 1,2,4 oxadiazole, compounds of formula IV may be prepared from compounds of formula VIII, where Y1 is a halogen, preferably bromine, as illustrated in Scheme 5. The procedure follows a cyclisation-condensation reaction with an hydroxyl amidine, such as hydroxyl acetamidine, and a suitable coupling reagent, such as T3P or HATU, in the presence of a base, such as triethylamine, in a suitable solvent, such as DMF at a temperature of 100° C. to 180° C.

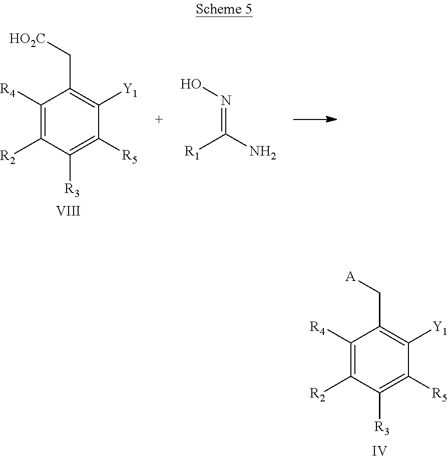

When A is oxazole compounds of formula IV may be prepared from compounds of formula VIII, where Y1 is a halogen, preferably bromine, as illustrated in Scheme 6.

Step i) Compounds of formula IX may be prepared from compounds of formula VIII by a coupling reaction with prop-2-yn-1-amine using a suitable coupling reagent such as T3P or HATU in the presence of an organic base, such as triethylamine, in a suitable solvent such as DMF, ethyl acetate or DCM at a temperature of 0° C. to 50° C., preferably at room temperature.

Step ii) Compounds of formula IV can be prepared from compound of formula IX by a cyclisation—condensation reaction under strong acid conditions, such as triflic acid, in a suitable solvent such as 1,4 dioxane, at a temperature of 50° C. to 120° C., preferably at 90° C. Those skilled in the art would appreciate that other annulation methods are known for the synthesis of an oxazole rings.

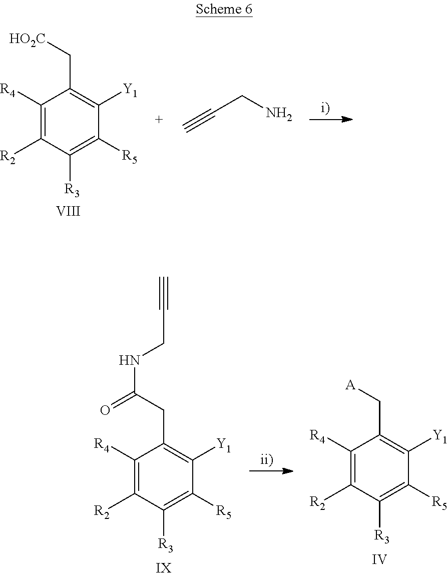

When A is 1,3,4 oxadiazole, compounds of formula IV may be prepared from compounds of formula VIII, where Y1 is a halogen, preferably a bromine, as illustrated in Scheme 7.

Step i) Compounds of formula X may be prepared from compounds of formula VIII by a coupling reaction with an acyl hydrazide, such as acetohydrazide, using a suitable coupling reagent such as T3P or HATU in the presence of an organic base such as triethylamine, in a suitable solvent such as DMF, ethyl acetate or DCM at a temperature of 0° C. to 50° C., preferably at room temperature.

Step ii) Compounds of formula IV can be prepared from compounds of formula X by a cyclisation—condensation reaction with a suitable reagent such as the Burgess reagent or triphenyl phosphine with hexachloroethane and a suitable organic base such as triethylamine. The reaction is performed in a suitable solvent such as THF or DCM at a temperature between 25° C. and solvent reflux, preferably at solvent reflux.

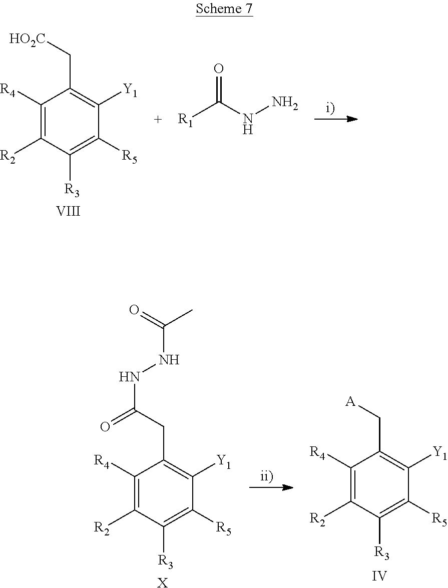

When A is the alternate 1,2,4 oxadiazole, compounds of formula IV may be prepared from compounds of formula VII where Y1 is a halogen, preferably bromine, as illustrated in Scheme 8.

Step i. Compounds of formula XI may be prepared from compounds of formula VII by a nucleophilic substitution reaction with a cyanide reagent, such as potassium cyanide, in a suitable solvent such as THF or DMF, at a temperature of 0° C. to 50° C., preferably at room temperature.

Step ii. Compounds of formula XII may be prepared from compounds of formula XI by a reaction with hydroxylamine in the presence of a base, such as potassium carbonate, in a suitable solvent such as ethanol at a temperature of 80° C. to 120° C., preferably 95° C.

Step iii. Compounds of formula XIII may be prepared from compounds of formula XII by an acylation reaction with a suitable acetylating reagent, such as acetyl chloride or a carboxylic acid together with a suitable coupling reagent, such as T3P or HATU in the presence of an organic base, such as triethylamine, in a suitable solvent such as DCM or THF, at a temperature of 0° C. to 50° C., preferably at room temperature. The acyl halide or carboxylic acid is either commercially available or synthesized from known procedures.

Step iv. Compounds of formula IV may be prepared from compounds of formula XIII by a cyclisation—condensation reaction using a suitable reagent such as hexachloroethane in an acidic solvent such acetic acid, at a temperature of 80° C. to 120° C., preferably at 100° C.

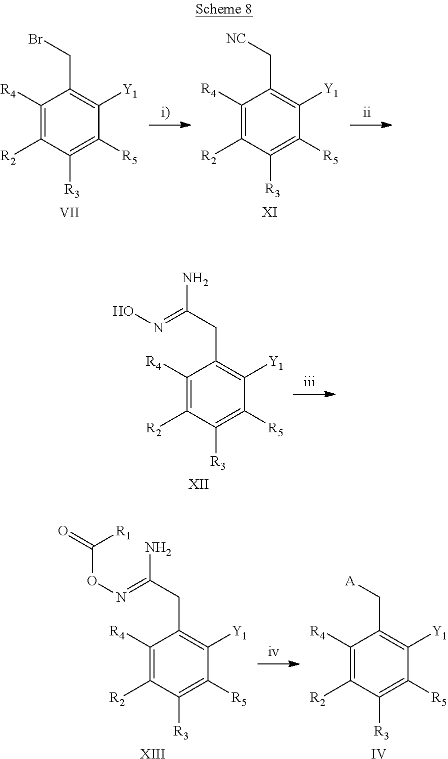

When A is a carbon linked tetrazole, compounds of formula IV may be prepared from compounds of formula XI, where Y1 is a halogen, preferably bromine, or an ester group, preferably methylester, as illustrated in Scheme 9.

Step i: Compounds of formula XV may be prepared from compounds of formula XI by reaction with an azide, such as sodium azide in a suitable solvent such as toluene at a temperature of 80° C. to 120° C., preferably at reflux temperature.

Step ii. Compounds of formula IV may be prepared from compounds of formula XV by alkylation with a suitable alkylating agent such as methyl iodide, in the presence of a suitable base, such triethylamine, in a suitable solvent such as DMF, or MeCN, at a temperature of 25° C. to 100° C., preferably 80° C.

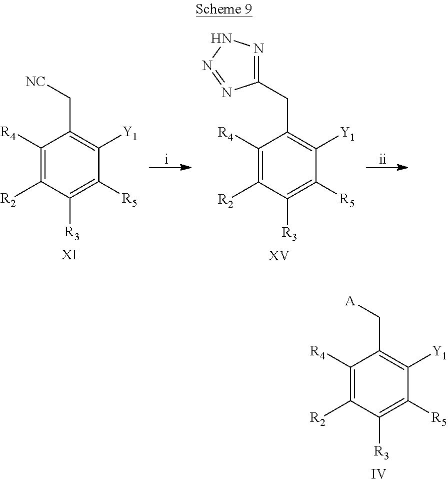

Compounds of formula VII may be prepared from compounds of formula XVI, where Y1 is a halogen, preferably bromine or an ester group, preferably methylester, by a bromination reaction illustrated in Scheme 10 using a suitable brominating agent, such as N-bromosuccinimide and a radical initiator such as AIBN in a suitable solvent such as tBuOAc at a temperature of 80° C. to 120° C., preferably at 90° C.

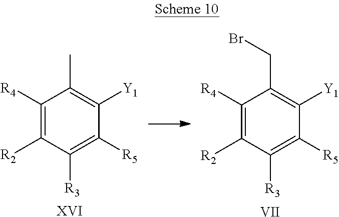

When Y1 is an ester, such as a methyl ester, compounds of formula XVI may be prepared from compounds of formula XVII as illustrated in Scheme 11.

Step 1. Compounds of formula XVIII may be prepared from compounds of formula XVII using methods described in the literature. G. P Lahm et. al. Bioorg. Med. Chem. Lett. 15 (2005) 4898-4906.

Step ii. Compounds of formula XVI may be prepared from compounds of formula XVIII by esterification in a suitable alcoholic solvent, such as methanol, in the presence of a strong acid, such as sulfuric acid, at reflux temperature.

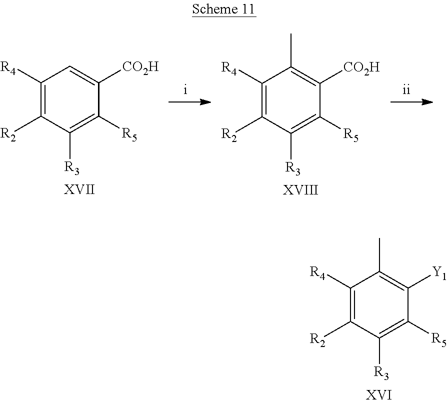

When Y is CH3CH═CH, Compounds of formula II, may be prepared from compounds of formula IV where Y1 is an ester, such as a methyl ester, as illustrated in Scheme 12.

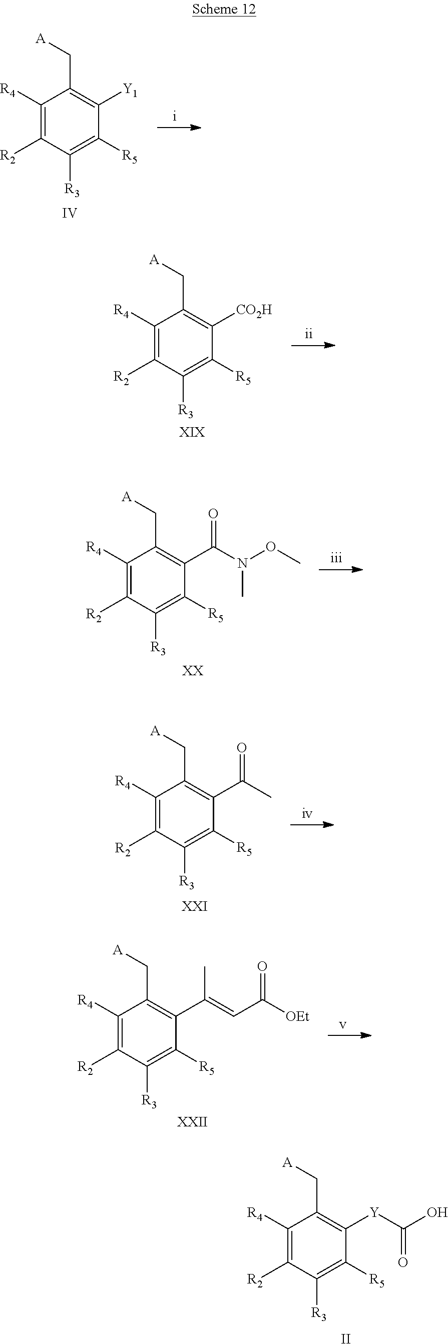

Step i. Compounds of formula XIX may be prepared from compounds of formula IV by standard saponification conditions known to those skilled in the art.

Step ii. Compounds of formula XX may be prepared from compounds of formula XIX by a coupling reaction with N,O-dimethylhydroxylamine using a suitable coupling reagent such as HATU or T3P in the presence of a suitable base, such as triethylamine, in a suitable solvent such as DMF or EtOAc, at a temperature of 0° C. to 50° C., preferably at room temperature.

Step iii. Compounds of formula XXI may be prepared from compounds of formula XX by a nucleophilic substitution reaction with a suitable Grignard reagent, such methylmagnesium bromide, in a suitable solvent such as THF or diethylether at a temperature of −78° C. to 0° C., preferably at −78° C.

Step iv. Compounds of formula XXII may be prepared from compounds of formula XXI by reaction with a suitable ylid such as triethylphosphonoacetate and sodium hydride, in a suitable solvent such as THF at a temperature of 0° C. to 60° C.

Step v. Compounds of formula II may be prepared from compounds of formula XXII by standard saponification conditions known to those skilled in the art.

When Y is CH2-O, Compounds of formula I, may be prepared from compounds of formula IV where Y1 is an ester, such as a methyl ester, as illustrated in Scheme 13.

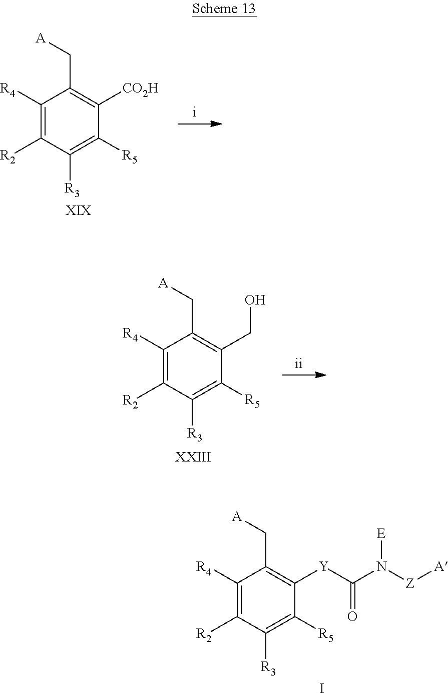

Step i. Compounds of formula XXIII may be prepared from compounds of formula XIX by reduction of the carboxylic acid group using a reducing agent such as borane THF complex in a suitable solvent such as THF, at a temperature of 25° C. to 50° C., preferably at 50° C.

Step ii. Compounds of formula I may be prepared from compounds of formula XXIII by a coupling reaction with compounds of formula III using a phosgene equivalent, such as triphosgene or carbonyl diimidazole, preferably carbonyl diimidazole, in a suitable solvent such as DMF at a temperature of 0° C. to 25° C., preferably 25° C.

When Y is —O—CH2-, compounds of formula II may be prepared from compounds of formula XXIVI as illustrated in Scheme 14.

Step i. Compounds of formula XXV may be prepared from compounds of formula XXIV by an alkylation reaction with a halo acetate, such as tert-butyl 2-bromoacetate, in the presence of a base, such as potassium carbonate, in a suitable solvent such as THF or MeCN at 25° C. to 50° C., preferably at room temperature.

Step ii. Compounds of formula XXVI may be prepared from compounds of formula XXV by a bromination reaction with a brominating agent, such as hexabromoacetone and triphenyl phosphine, in a suitable solvent, such as MeCN, at 25° C. to 80° C., preferably at 40° C.

Step iii. Compounds of formula II may be prepared from compounds of formula XXVI, by chemistry illustrated in scheme 4, where Y1 is —O—CH2-CO2tBu.

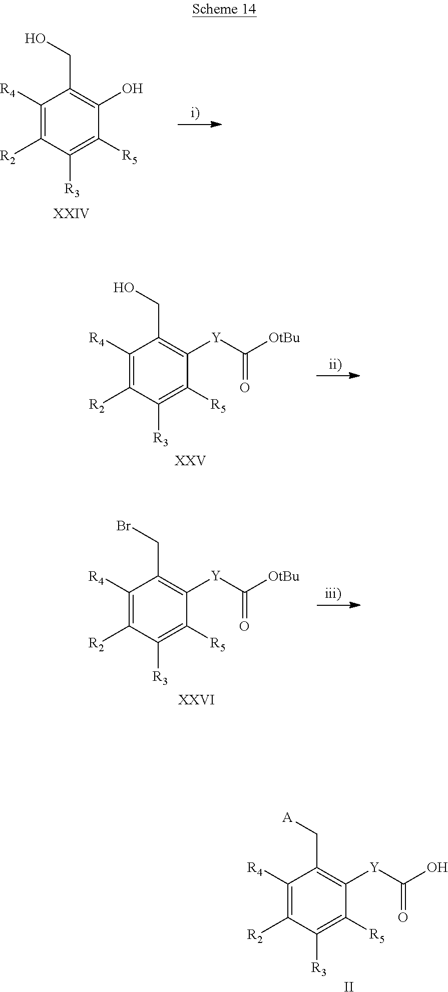

Compounds of formula III are either commercially available or synthesized by known procedures, or by the following schemes where P is a protecting group. Protection and deprotection strategies are known to those skilled in the art, examples of which can be found in the publication ‘Protective Groups in Organic Synthesis’ by Green et al. Unless otherwise stated, starting materials are either commercially available or synthesized by known procedures.

Compounds of formula I, may be prepared from compounds of formula II as illustrated in scheme 15 and 16

Step i. Compounds of formula XXVII, may be prepared from compounds of formula II where Y is —CH═CH—, —CH3CH═CH—, —CH2-CH2- or —O—CH2- by a coupling reaction with either protected N-methyl-1-(piperidin-4-yl)methanamine, such as tert-butyl 4-((methylamino)methyl)piperidine-1-carboxylate or protected N-methylpiperidin-4-amine such as tert-butyl 4-(methylamino)piperidine-1-carboxylate with a suitable coupling reagent such as HATU or T3P in the presence of a suitable base, such as triethylamine, in a suitable solvent such as DMF or EtOAc, at a temperature of 0° C. to 50° C., preferably at room temperature.

Step ii. Compounds of formula I may be prepared from compounds of formula XXVII or XXVIII by reaction with a suitable sulfonyl chloride in the presence of an organic base, such as triethylamine, in a suitable solvent such as DMF or DCM, or by reaction with a carboxylic acid and a suitable coupling reagent such as T3P or HATU, in a suitable solvent such as DMF or EtOAc or by reaction with a suitable aldehyde or ketone, in the presence of a reducing agent, such as sodium triacetoxyborohydride, or picoline borane, in a suitable solvent such as THF or MeOH/acetic acid at a temperature of 25° C. to 50° C., preferably at room temperature.

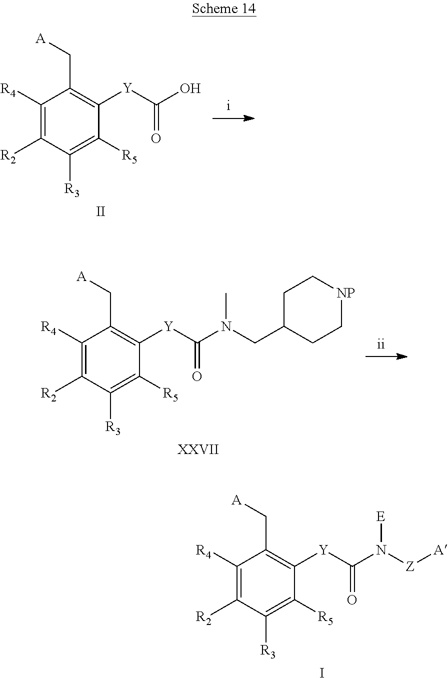

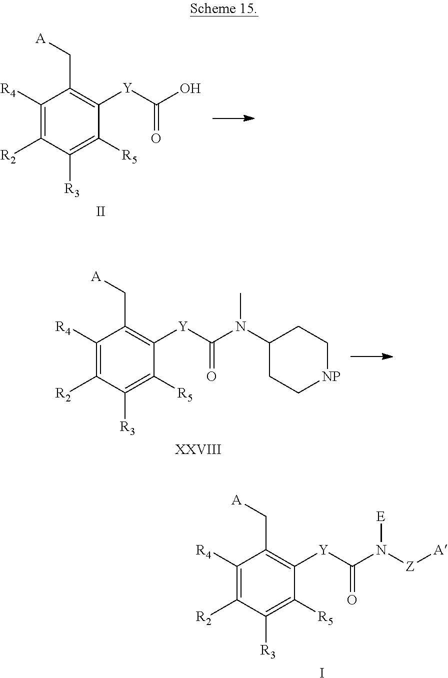

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compounds of the present invention. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds of the present invention described herein may contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds of the present invention may be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound of the present invention contains a double bond, the substituent may be E or Z configuration. If the compound of the present invention contains a disubstituted cycloalkyl, the cycloalkyl substituent may have a cis- or trans-configuration. All tautomeric forms, for example for group A in embodiment 1, are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of the present invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the compounds of the present invention can be synthesized from a basic or acidic moiety, by conventional chemical methods. Generally, such salts can be prepared by reacting free acid forms of the compounds of the present invention with a stoichiometric amount of the appropriate base (such as Na, Ca, Mg, or K hydroxide, carbonate, bicarbonate or the like), or by reacting free base forms of the compounds of the present invention with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds of the present invention. Isotopically labeled compounds of the present invention have structures depicted by the formulas given herein except that one or more atoms are replaced by an atom having a selected atomic mass or mass number. Examples of isotopes that can be incorporated into compounds of the present invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, 35S, ³⁶Cl, ¹²⁵I respectively. The invention includes various isotopically labeled compounds of the present invention, for example those into which radioactive isotopes, such as ³H and ¹⁴C, or those into which non-radioactive isotopes, such as ²H and ¹³C are present. Such isotopically labelled compounds of the present invention are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound of the present invention may be particularly desirable for PET or SPECT studies. Isotopically-labeled compounds of the present invention can generally be prepared by conventional techniques known to those skilled in the art or by processes analogous to those described in the accompanying Generic Schemes, Examples and Preparations using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of the present invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

The compounds of the present invention in free form or in salt form, exhibit valuable pharmacological properties, e.g. as indicated in in vitro tests as provided herein, and are therefore indicated for therapy or for use as research chemicals, e.g. as tool compounds.

Thus, in an embodiment 16, there is provided a compound according to any one of embodiments 1 to 15 for use in medicine.

The compounds according to any one of embodiments 1 to 15 are potent inhibitors of ATX (see IC₅₀ data disclosed herein). The compounds of the present invention are hence useful in the treatment of an ATX-dependent or ATX-mediated disease or condition.

Thus, in an embodiment 17, there is provided a compound according to any one of embodiments 1 to 15 for use in the treatment of an ATX-dependent or ATX-mediated disease or condition. In an embodiment 18, there is provided the use of a compound according to any one of embodiments 1 to 15 in the treatment of an ATX-dependent or ATX-mediated disease or condition. In an embodiment 19, there is provided the use of a compound according to any one of embodiments 1 to 15 in the manufacture of a medicament for the treatment of an ATX-dependent or ATX-mediated disease or condition. In an embodiment 20, there is provided a method of treating an ATX-dependent or ATX-mediated disease or condition comprising administering to the subject a therapeutically effective amount of a compound according to any one of embodiments 1 to 15.

Hence, in a further embodiment 21, the compounds of the invention are useful for the treatment of a disease or condition according to embodiments 17, 18, 19 and 20, wherein the disease or condition is selected from fibrosis, pruritus, cirrhosis, cancer, diabetes, kidney diseases and pain.

In an embodiment 22, the compounds of the invention are useful for the treatment of a disease or condition according to embodiment 21, wherein the disease or condition is selected from pulmonary fibrosis, idiopathic pulmonary fibrosis, a diffuse parenchymal interstitial lung disease including iatrogenic drug-induced fibrosis, occupational and/or environmental induced fibrosis (Farmer lung), radiation induced fibrosis, bleomycin induced pulmonary fibrosis, asbestos induced pulmonary fibrosis, acute respiratory distress syndrome (ARDS), kidney fibrosis, tubulointerstitium fibrosis, gut fibrosis, liver fibrosis, alcohol induced liver fibrosis, toxic/drug induced liver fibrosis, infection induced liver fibrosis, viral induced liver fibrosis, cutaneous fibrosis, spinal cord injury/fibrosis, myelofibrosis, renal fibrosis, skin fibrosis, ocular fibrosis, post-transplant fibrosis, hepatic fibrosis with or without cirrhosis, cardiac fibrosis, neuropathic pruritus, neurogenic pruritus, psychogenic pruritus, cholestatic pruritus, primary biliary cirrhosis, liver cirrhosis, breast cancer, pancreatic cancer, ovarian cancer, prostate cancer, glioblastoma, bone cancer, colon cancer, bowel cancer, head and neck cancer, diabetes, polycystic kidney disease, acute kidney injury, chronic kidney disease, neuropathic pain and cancer pain.

In an embodiment 23, the compounds of the invention are useful for the treatment of a disease or condition according to embodiment 22, wherein the disease or condition is selected from idiopathic pulmonary fibrosis, breast cancer, pancreatic cancer, prostate cancer, cholestatic pruritus, primary biliary cirrhosis and polycystic kidney disease, particularly idiopathic pulmonary fibrosis.

The compounds of the invention will be typically formulated as pharmaceutical compositions.

Thus, in an embodiment 24 of the invention, the present invention provides a pharmaceutical composition comprising a compound according to any one of embodiments 1 to 15, or a pharmaceutically acceptable salt thereof, and a pharmaceutically acceptable carrier.

The pharmaceutical composition can be formulated for particular routes of administration such as oral administration, parenteral administration, and rectal administration, inhaled administration etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the present invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multidose dry powder inhalation (MDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate. Suitable such dry powder inhalation devices include devices disclosed in U.S. Pat. No. 3,991,761 (including the AEROLIZER™ device), WO 05/113042 (including the BREEZHALER™ device), WO 97/20589 (including the CERTIHALER™ device), WO 97/30743 (including the TWISTHALER™ device), WO 05/37353 (including the GYROHALER™ device), U.S. Pat. No. 6,536,427 (including the DISKUS™ device), WO 97/25086 (including the DISKHALER™ device), WO 95/14089 (including the GEMINI™ device), WO 03/77979 (including the PROHALER™ device), and also the devices disclosed in WO 08/51621, WO 09/117112 and US 2005/0183724.

Hence, the invention also includes (A) a compound of the present invention, or a pharmaceutically acceptable salt thereof, in inhalable form; (B) an inhalable medicament comprising a compound of the present invention in inhalable form together with a pharmaceutically acceptable carrier in inhalable form; (C) a pharmaceutical product comprising a compound of the present invention in inhalable form in association with an inhalation device; and (D) an inhalation device containing a compound of the present invention in inhalable form.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of the present invention and at least one other therapeutic agent as a combined preparation for simultaneous, separate or sequential use in therapy. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

Thus, in an embodiment 25, the invention provides a pharmaceutical composition comprising a compound according to any one of embodiments 1 to 15 and one or more therapeutically active co-agent. Optionally, the pharmaceutical composition may comprise a pharmaceutically acceptable excipient, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In an embodiment 26 of the invention, there is provided a pharmaceutical combination, comprising:

a therapeutically effective amount of the compound according to any one of embodiments 1 to 15, or a pharmaceutically acceptable salt thereof, and one or more therapeutically active co-agent.

In an embodiment 27 of the invention, there is provided a pharmaceutical combination according to embodiment 26, wherein the therapeutically active co-agent is selected from immunosuppresants, analgesics, anti-cancer agent, anti-inflammatories, chemokine receptor antagonists, bronchodilators, leukotriene receptor antagonists, leukotriene formation inhibitors, monoacylglycerol kinase inhibitors, phospholipase A1 inhibitors, phospholipase A2 inhibitors, lysophospholipase D (lysoPLD) inhibitors, decongestants, antihistamines, mucolytics, anticholinergics, antitussives, expectorants, and β2 agonists.

Suitable anti-inflammatory drugs include steroids, for example corticosteroids. Suitable steroids include budesonide, beclamethasone (e.g. dipropionate), butixocort (e.g. propionate), CHF5188, ciclesonide, dexamethasone, flunisolide, fluticasone (e.g. propionate or furoate), GSK-685698, GSK-870086, LAS40369, methyl prednisolone, mometasone (e.g. furoate), prednisolone, rofleponide, and triamcinolone (e.g. acetonide). In certain preferred embodiments the steroid is long-acting corticosteroids such as budesonide, ciclesonide, fluticasone propionate, fluticasone furoate or mometasone furoate.

Suitable β2-agonists include arformoterol (e.g. tartrate), albuterol/salbutamol (e.g. racemate or single enantiomer such as the R-enantiomer, or salt thereof especially sulfate), AZD3199, bambuterol, BI-171800, bitolterol (e.g. mesylate), carmoterol, clenbuterol, etanterol, fenoterol (e.g. racemate or single enantiomer such as the R-enantiomer, or salt thereof especially hydrobromide), flerbuterol, formoterol (e.g. racemate or single diastereomer such as the R,R-diastereomer, or salt thereof especially fumarate or fumarate dihydrate), GSK-159802, GSK-597901, GSK-678007, indacaterol (e.g. racemate or single enantiomer such as the R-enantiomer, or salt thereof especially maleate, acetate or xinafoate), LAS100977, metaproterenol, milveterol (e.g. hydrochloride), naminterol, olodaterol (e.g. racemate or single enantiomer such as the R-enantiomer, or salt thereof especially hydrochloride), PF-610355, pirbuterol (e.g. acetate), procaterol, reproterol, salmefamol, salmeterol (e.g. racemate or single enantiomer such as the R-enantiomer, or salt thereof especially xinafoate), terbutaline (e.g. sulphate) and vilanterol (or a salt thereof especially trifenatate. In certain preferred embodiments the β₂-agonist is an ultra-long-acting β₂-agonist such as indacaterol, or potentially carmoterol, LAS-100977, milveterol, olodaterol, PF-610355 or vilanterol. A preferred embodiment one of the second active ingredients is indacaterol (i.e. (R)-5-[2-(5,6-diethyl-indan-2-ylamino)-1-hydroxyethyl]-8-hydroxy-1H-quinolin-2-one) or a salt thereof. This is a β₂-adrenoceptor agonist that has an especially long duration of action (i.e. over 24 hours) and a short onset of action (i.e. about 10 minutes). This compound is prepared by the processes described in international patent applications WO 2000/75114 and WO 2005/123684. It is capable of forming acid addition salts, particularly pharmaceutically acceptable acid addition salts. A preferred salt of (R)-5-[2-(5,6-diethyl-indan-2-ylamino)-1-hydroxyethyl]-8-hydroxy-1H-quinolin-2-one is the maleate salt. Another preferred salt is (R)-5-[2-(5,6-diethyl-indan-2-ylamino)-1-hydroxyethyl]-8-hydroxy-1H-quinolin-2-one acetate. Another preferred salt is (R)-5-[2-(5,6-diethyl-indan-2-ylamino)-1-hydroxyethyl]-8-hydroxy-1H-quinolin-2-one xinafoate.

Suitable bronchodilatory drugs include anticholinergic or antimuscarinic agents, such as aclidinium (e.g. bromide), BEA-2108 (e.g. bromide), BEA-2180 (e.g. bromide), CHF-5407, darifenacin (e.g. bromide), darotropium (e.g. bromide), glycopyrrolate (e.g. racemate or single enantiomer, or salt thereof especially bromide), dexpirronium (e.g. bromide), iGSK-202405, GSK-203423, GSK-573719, GSK-656398, ipratropium (e.g. bromide), LAS35201, LAS186368, otilonium (e.g. bromide), oxitropium (e.g. bromide), oxybutynin, PF-3715455, PF-3635659, pirenzepine, revatropate (e.g. hydrobromide), solifenacin (e.g. succinate), SVT-40776, TD-4208, terodiline, tiotropium (e.g. bromide), tolterodine (e.g. tartrate), and trospium (e.g. chloride). In certain preferred embodiments the muscarinic antagonists is long-acting muscarinic antagonist such as darotropium bromide, glycopyrrolate or tiotropium bromide.

Suitable dual anti-inflammatory and bronchodilatory drugs include dual beta-2 adrenoceptor agonist/muscarinic antagonists such as GSK-961081 (e.g. succinate). and those disclosed in USP 2004/0167167, WO 04/74246 and WO 04/74812.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

### EXPERIMENTAL

### Examples

The following examples are intended to illustrate the invention and are not to be construed as being limitations thereon.

### General Conditions:

Mass spectra were acquired on LC-MS, SFC-MS, or GC-MS systems using electrospray, chemical and electron impact ionization methods from a range of instruments of the following configurations: Agilent 1100 HPLC systems with an Agilent 6110 Mass Spectrometer [M+H]+ refers to protonated molecular ion of the chemical species.

NMR spectra were run on Bruker AVANCE 400 MHz or 500 MHz NMR spectrometers using ICON-NMR, under TopSpin program control. Spectra were measured at 298K, unless indicated otherwise, and were referenced relative to the solvent resonance.

### Instrumentation:

MS Methods: Using Agilent 1100 HPLC systems with an Agilent 6110 Mass Spectrometer

2minLowpHv01:
Column: Waters Acquity CSH 1.7 μm, 2.1×50 mm
Temperature: 50° C.
Mobile Phase: A: Water+0.1% Formic Acid B: Acetonitrile+0.1% Formic Acid
Flow rate: 1.0 mL/min
Gradient: 0.0 min 5% B, 0.2-1.55 min 5-98% B, 1.55-1.75 min 98% B, 1.75-1.8 min 98-5% B
   2minLowpHv02:
Column: Waters Acquity CSH 1.7 μm, 2.1×50 mm
Temperature: 50° C.
Mobile Phase: A: Water+0.1% TFA B: Acetonitrile+0.1% TFA
Flow rate: 1.0 mL/min
Gradient: 0.0 min 5% B, 0.2-1.55 min 5-98% B, 1.55-1.75 min 98% B, 1.75-1.8 min 98% B
   2minLowpHv03:
Column: Waters Acquity CSH 1.7 μm, 2.1×50 mm
Temperature: 50° C.
Mobile Phase: A: Water+0.1% Formic Acid B: Acetonitrile+0.1% Formic Acid
Flow rate: 1.0 mL/min
Gradient: 0.0 min 5% B, 0.2-1.8 min 5-98% B, 1.8-2.1 min 98% B, 2.1-2.3 min 98% B
   8minLowpHv01:
Column: Waters Acquity CSH 1.7 μm, 2.1×100 mm
Temperature: 50° C.
Mobile Phase: A: Water+0.1% Formic Acid B: Acetonitrile+0.1% Formic Acid
Flow rate: 0.7 mL/min
Gradient: 0.0 min 2% B, 0.3-6.5 min 2-98% B, 6.5-7.5 min 98% B, 7.5-8.0 min 5-98% B

### ABBREVIATIONS

BOC tertiary butyl carboxy
br broad
d doublet
dd doublet of doublets
DCM dichloromethane
DIPEA diethylisopropylamine
DMA N,N-dimethylformamide
DME 1,4-dimethoxyethane
DMF N,N-dimethylformamide
DMSO dimethylsulfoxide
EtOAc ethyl acetate
Et₃N triethylamine
h hour(s)
HPLC high pressure liquid chromatography
LCMS liquid chromatography and mass spectrometry
MeOH methanol
MS mass spectrometry
m or mult multiplet
mg milligram
min minutes
mL milliliter
mmol millimol
m/z mass to charge ratio
NMR nuclear magnetic resonance
ppm parts per million
rac racemic
Rt retention time
s singlet
t triplet
TFA trifluoroacetic acid

### Preparation of Final Compounds

### Example 1.1

### (E)-tert-Butyl 4-((3-(4-chloro-2-((5-methyl-2H-tetrazol-2-yl)methyl)phenyl)-N-methylacrylamido)methyl)piperidine-1-carboxylate

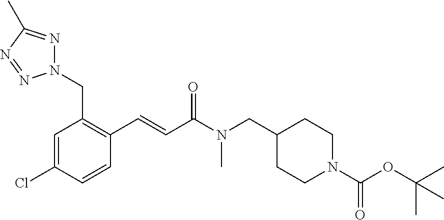 [SMILES: Cc1nnn(Cc2cc(Cl)ccc2/C=C/C(=O)N(C)CC2CCN(C(=O)OC(C)(C)C)CC2)n1]

(E)-3-(4-Chloro-2-((5-methyl-2H-tetrazol-2-yl)methyl)phenyl)acrylic acid (Intermediate A) (200 mg, 0.718 mmol) and tert-butyl 4-((methylamino)methyl)piperidine-1-carboxylate (154 mg, 0.718 mmol) were dissolved in DMF (3 ml). DIPEA (501 ul, 2.87 mmol) was added followed by 50% T3P® solution in DMF (838 ul, 1.435 mmol) and the mixture was stirred at room temperature overnight. The resulting mixture was concentrated in vacuo. The residue was dissolved in EtOAc and washed with 10% citric acid, saturated bicarbonate solution, brine, dried over MgSO4, filtered and concentrated in vacuo. Purification by chromatography on silica eluting with a gradient of EtOAc in iso-hexane afforded the title compound;

LCMS: Rt 1.26 mins MS m/z 389.5 [M+H-Boc]+ Method 2minLowpHv01.

### Example 1.2

### (E)-tert-Butyl-4-(N-ethyl-3-(2-((5-methyl-2H-tetrazol-2-yl)methyl)-4-(trifluoromethyl)phenyl)acrylamido)piperidine-1-carboxylate

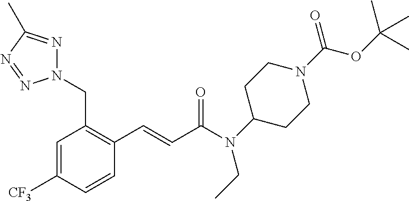 [SMILES: CCN(C(=O)/C=C/c1ccc(C(F)(F)F)cc1Cn1nnc(C)n1)C1CCN(C(=O)OC(C)(C)C)CC1]

The title compound was prepared from (E)-3-(2-((5-methyl-2H-tetrazol-2-yl)methyl)-4-(trifluoromethyl)phenyl)acrylic acid (Intermediate AB) and tert-butyl 4-(ethylamino)piperidine-1-carboxylate by a similar method to Example 1.1;

LCMS: Rt=1.47 mins; MS m/z 523.3 [M+H]+; Method 2minLowpHv03.

### Example 2.1

### (E)-3-(4-Chloro-2-((5-methyl-2H-tetrazol-2-yl)methyl)phenyl)-N-ethyl-N-(pyridin-4-ylmethyl)acrylamide

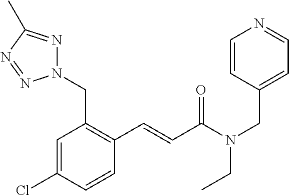 [SMILES: CCN(Cc1ccncc1)C(=O)/C=C/c1ccc(Cl)cc1Cn1nnc(C)n1]

A vial comprising (E)-3-(4-chloro-2-((5-methyl-2H-tetrazol-2-yl)methyl)phenyl)acrylic acid (Intermediate A) (0.028 g, 0.1 mmol) was treated with a solution of HATU (0.046 g, 0.120 mmol) in DMF (1 ml) and the mixture was stirred at room temperature. After 30 min, a solution of N-(pyridin-4-ylmethyl)ethanamine (0.014 g, 0.1 mmol) in DMF (1 ml) and Et₃N (0.028 ml, 0.200 mmol) was added and the vial was capped and shaken at room temperature for 72 hours.

The resulting product mixture was diluted with MeOH (1.5 ml) concentrated in vacuo. The residue was dissolved in DMSO (1 ml) and purified by reverse phase preparative HPLC. The product fractions were concentrated in vacuo, dissolved in MeOH (2 ml) and passed through a 2 g Biotage Si—CO3 cartridge washing through with MeOH (4×2 ml). The solvent was removed in vacuo to afford the title compound;

LCMS: Rt 0.83 mins; MS m/z 397.1[M+H]+; Method 2minLowpHv02

Examples 2.2 to 2.7 were prepared by a similar method to Example 2.1 by replacing N-(pyridin-4-ylmethyl)ethanamine with the appropriate commercially available amine:

### Example 2.2

### (E)-3-(4-Chloro-2-((5-methyl-2H-tetrazol-2-yl)methyl)phenyl)-N-methyl-N-((4-methylthiazol-2-yl)methyl)acrylamide

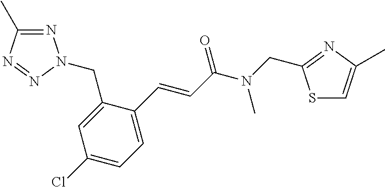

LCMS: Rt 1.05. mins; MS m/z 403.1[M+H]+; Method 2minLowpHv02

### Example 2.3

### (E)-3-(4-Chloro-2-((5-methyl-2H-tetrazol-2-yl)methyl)phenyl)-N-methyl-N-((3-methylisoxazol-5-yl)methyl)acrylamide

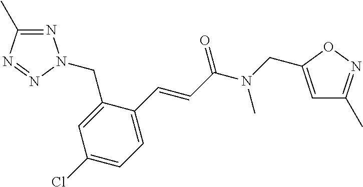

LCMS: Rt 1.06 mins; MS m/z 387.1[M+H]+; Method 2minLowpHv02

### Example 2.4

### (E)-3-(4-Chloro-2-((5-methyl-2H-tetrazol-2-yl)methyl)phenyl)-N-ethyl-N-(2-methoxyethyl)acrylamide

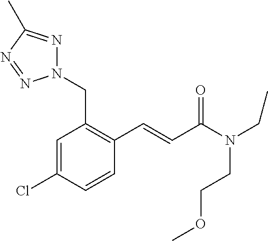 [SMILES: CCN(CCOC)C(=O)/C=C/c1ccc(Cl)cc1Cn1nnc(C)n1]

LCMS: Rt 1.07. mins; MS m/z 442.2[M+H]+; Method 2minLowpHv02

### Example 2.5

### (E)-3-(4-Chloro-2-((5-methyl-2H-tetrazol-2-yl)methyl)phenyl)-N-ethyl-N-(2-hydroxy-2-(3-hydroxyphenyl)ethyl)acrylamide

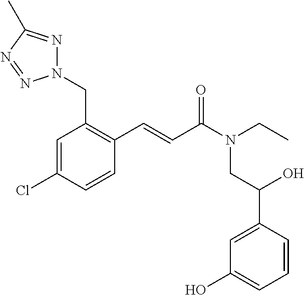 [SMILES: CCN(CC(O)c1cccc(O)c1)C(=O)/C=C/c1ccc(Cl)cc1Cn1nnc(C)n1]

LCMS: Rt 1.04 mins; MS m/z 364.1[M+H]+; Method 2minLowpHv02

### Example 2.6

### (E)-N-((1-(tert-Butyl)-5-oxopyrrolidin-3-yl)methyl)-3-(4-chloro-2-((5-methyl-2H-tetrazol-2-yl)methyl)phenyl)-N-methylacrylamide

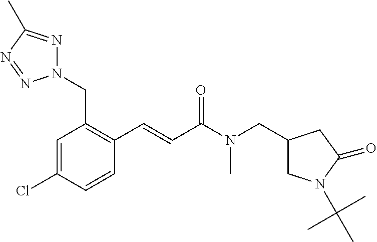 [SMILES: Cc1nnn(Cc2cc(Cl)ccc2/C=C/C(=O)N(C)CC2CC(=O)N(C(C)(C)C)C2)n1]

LCMS: Rt 1.08 mins; MS m/z 445.2[M+H]+; Method 2minLowpHv02

### Example 2.7

### (E)-3-(4-Chloro-2-((5-methyl-2H-tetrazol-2-yl)methyl)phenyl)-N-methyl-N-((tetrahydrofuran-2-yl)methyl)acrylamide

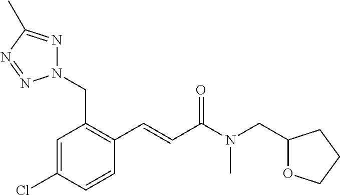 [SMILES: Cc1nnn(Cc2cc(Cl)ccc2/C=C/C(=O)N(C)CC2CCCO2)n1]

LCMS: Rt 1.05 mins; MS m/z 376.1[M+H]+; Method 2minLowpHv02

### Example 3

### (E)-N-((1-(4-Fluorobenzoyl)piperidin-4-yl)methyl)-N-methyl-3-(2-((5-methyl-2H-tetrazol-2-yl)methyl)-4-(trifluoromethyl)phenyl)acrylamide

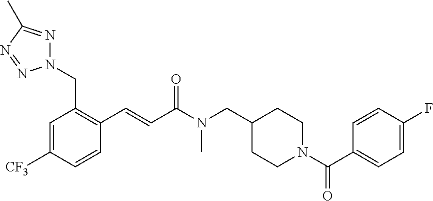 [SMILES: Cc1nnn(Cc2cc(C(F)(F)F)ccc2/C=C/C(=O)N(C)CC2CCN(C(=O)c3ccc(F)cc3)CC2)n1]

To (E)-N-methyl-3-(2-((5-methyl-2H-tetrazol-2-yl)methyl)-4-(trifluoromethyl)phenyl)-N-(piperidin-4-ylmethyl)acrylamide hydrochloride (Intermediate B) (100 mg, 0.218 mmol) and 4-fluorobenzoic acid (30.5 mg, 0.218 mmol) in DCM (2 mL) was added triethylamine (0.152 mL, 1.090 mmol) followed by T3P® (50% in DMF) (0.153 mL, 0.261 mmol) dropwise. After stirring at room temperature for 2 hours, the product mixture was diluted with EtOAc and washed with water and brine. The organic portion was dried using a phase separating column and concentrated in vacuo. Purification by chromatography on silica eluting with 0-100% EtOAc in iso-hexane followed by 10% MeOH in EtOAc afforded the title compound; LCMS: Rt=1.31 mins; MS m/z 545.3 [M+H]+; Method 2minLowpHv03.

### Example 4.1

### (E)-N-((1-(Cyclopropylsulfonyl)piperidin-4-yl)methyl)-N-methyl-3-(2-((5-methyl-2H-tetrazol-2-yl)methyl)-4-(trifluoromethyl)phenyl)acrylamide

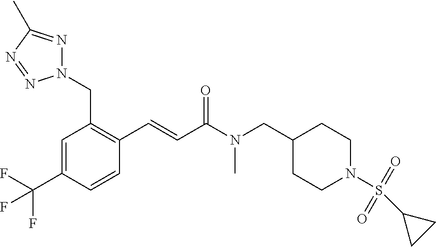 [SMILES: Cc1nnn(Cc2cc(C(F)(F)F)ccc2/C=C/C(=O)N(C)CC2CCN(S(=O)(=O)C3CC3)CC2)n1]

To (E)-N-methyl-3-(2-((5-methyl-2H-tetrazol-2-yl)methyl)-4-(trifluoromethyl)phenyl)-N-(piperidin-4-ylmethyl)acrylamide hydrochloride (Intermediate B) (101 mg, 0.163 mmol) in DCM (2 mL) was added triethylamine (0.146 mL, 1.046 mmol) followed by cyclopropanesulfonyl chloride (0.071 mL, 0.697 mmol) and the mixture was stirred at room temperature for 3 h. The product mixture was diluted with DCM and washed with water. The organic portion was separated, dried using a phase separating column and concentrated in vacuo. Purification by chromatography on silica eluting with 0-100% EtOAc in iso-hexane afforded a solid which was dissolved in DCM and concentrated in vacuo to afford the title compound;

LCMS: Rt=1.26 mins; MS m/z 527.7 [M+H]+; Method 2minLowpHv03.

### Example 4.2

### (E)-N-Methyl-3-(2-((5-methyl-2H-tetrazol-2-yl)methy)-4-(trifluoromethyl)phenyl)-N-((1-tosylpiperidin-4-yl)methyl)acrylamide

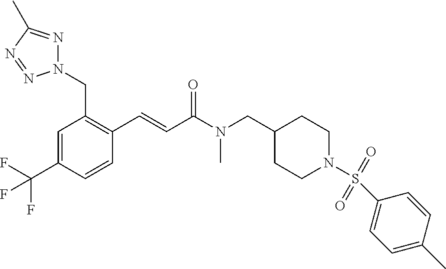 [SMILES: Cc1ccc(S(=O)(=O)N2CCC(CN(C)C(=O)/C=C/c3ccc(C(F)(F)F)cc3Cn3nnc(C)n3)CC2)cc1]

The title compound was prepared by a similar method to Example 4.1 from (E)-N-methyl-3-(2-((5-methyl-2H-tetrazol-2-yl)methyl)-4-(trifluoromethyl)phenyl)-N-(piperidin-4-ylmethyl)acrylamide hydrochloride (Intermediate B) and 4-fluorobenzaldehyde; LCMS: Rt=1.42 mins; MS m/z 577.7 [M+H]+; Method 2minLowpHv03.

### Example 5.0

### (E)-N-((1-(4-Fluorobenzyl)piperidin-4-yl)methyl)-N-methyl-3-(2-((5-methyl-2H-tetrazol-2-yl)methyl)-4-(trifluoromethyl)phenyl)acrylamide

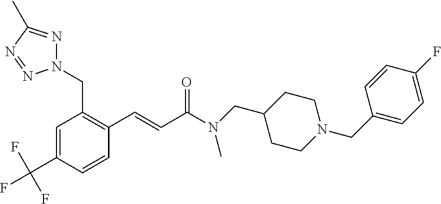 [SMILES: Cc1nnn(Cc2cc(C(F)(F)F)ccc2/C=C/C(=O)N(C)CC2CCN(Cc3ccc(F)cc3)CC2)n1]

To (E)-N-methyl-3-(2-((5-methyl-2H-tetrazol-2-yl)methyl)-4-(trifluoromethyl)phenyl)-N-(piperidin-4-ylmethyl)acrylamide hydrochloride (Intermediate B) (100 mg, 0.218 mmol) in MeOH (1 mL) and acetic acid (0.1 mL) was added 4-fluorobenzaldehyde (0.035 mL, 0.327 mmol) and the mixture was stirred for 5 min. 2-Picoline borane (36.9 mg, 0.349 mmol) was added and the resulting mixture was stirred at room temperature overnight. The solvent was removed in vacuo and the crude product was purified by reverse phase chromatography. The product fractions were concentrated in vacuo and the residue was re-dissolved in DCM and concentrated to afford the title compound;

LCMS: Rt=0.94 mins; MS m/z 531.6 [M+H]+; Method 2minLowpHv03.

### Example 6.1

### (E)-N-((1,3-Dimethyl-1H-pyrazol-5-yl)methyl)-N-methyl-3-(2-((5-methyl-2H-tetrazol-2-yl)methyl)-4-(trifluoromethyl)phenyl)acrylamide

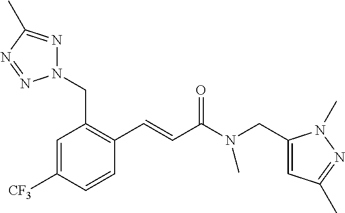 [SMILES: Cc1cc(CN(C)C(=O)/C=C/c2ccc(C(F)(F)F)cc2Cn2nnc(C)n2)n(C)n1]

DMF (1 ml) was added to 1-(1,3-dimethyl-1H-pyrazol-5-yl)-N-methylmethanamine (22.29 mg, 0.160 mmol) followed by triethylamine (0.067 ml, 0.480 mmol) and the mixture was stirred at room temperature.

In a separate flask, (E)-3-(2-((5-methyl-2H-tetrazol-2-yl)methyl)-4-(trifluoromethyl)phenyl)acrylic acid (Intermediate AB) (50 mg, 0.160 mmol) in DMF (1 ml) was treated with HATU (73.1 mg, 0.192 mmol) and stirred for 5 minutes. The resulting solution was pipetted into the pre-stirred amine solution and the reaction mixture was stirred at room temperature overnight. The solvent was removed in vacuo and the crude product was purified by reverse phase chromatography. The product fractions were concentrated in vacuo and the residue was re-dissolved in DCM and concentrated to afford the title compound;

LCMS: Rt=1.17 mins; MS m/z 434.9 [M+H]+; Method 2minLowpHv03.

Example 6.2-6.10 were prepared by a similar method to Example 6.1 from (E)-3-(2-((5-methyl-2H-tetrazol-2-yl)methyl)-4-(trifluoromethyl)phenyl)acrylic acid (Intermediate AB) and the commercially available amine. The reaction was carried out in either DMF or DMA:

### Example 6.2

### (E)-N-Methyl-3-(2-((5-methyl-2H-tetrazol-2-yl)methyl)-4-(trifluoromethyl)phenyl)-N-(2-phenoxyethyl)acrylamide

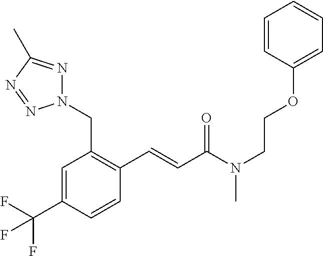 [SMILES: Cc1nnn(Cc2cc(C(F)(F)F)ccc2/C=C/C(=O)N(C)CCOc2ccccc2)n1]

LCMS: Rt=1.43 mins; MS m/z 446.3 [M+H]+; Method 2minLowpHv03.

### Example 6.3

### (E)-N-Methyl-3-(2-((5-methyl-2H-tetrazol-2-yl)methyl)-4-(trifluoromethyl)phenyl)-N-((4-methylthiazol-2-yl)methyl)acrylamide

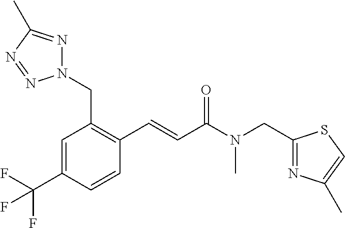 [SMILES: Cc1csc(CN(C)C(=O)/C=C/c2ccc(C(F)(F)F)cc2Cn2nnc(C)n2)n1]

LCMS: Rt=1.26 mins; MS m/z 437.8 [M+H]+; Method 2minLowpHv03.

### Example 6.4

### (E)-N-Methyl-3-(2-((5-methyl-2H-tetrazol-2-yl)methyl)-4-(trifluoromethyl)phenyl)-N-((5-phenyl-1,3,4-oxadiazol-2-yl)methyl)acrylamide

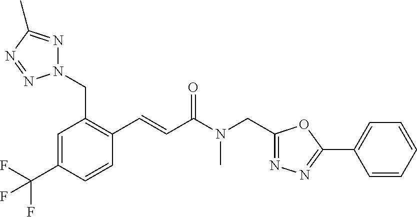 [SMILES: Cc1nnn(Cc2cc(C(F)(F)F)ccc2/C=C/C(=O)N(C)Cc2nnc(-c3ccccc3)o2)n1]

LCMS: Rt=1.31 mins; MS m/z 484.2 [M+H]+; Method 2minLowpHv03.

### Example 6.5

### (E)-N-(2-((4-Methoxybenzyl)amino)-2-oxoethyl)-N-methyl-3-(2-((5-methyl-2H-tetrazol-2-yl)methyl)-4-(trifluoromethyl)phenyl)acrylamide

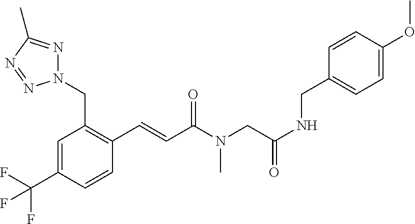 [SMILES: COc1ccc(CNC(=O)CN(C)C(=O)/C=C/c2ccc(C(F)(F)F)cc2Cn2nnc(C)n2)cc1]

LCMS: Rt=1.23 mins; MS m/z 503.3 [M+H]+; Method 2minLowpHv03.

### Example 6.6

### (E)-N-Methyl-3-(2-((5-methyl-2H-tetrazol-2-yl)methyl)-4-(trifluoromethyl)phenyl)-N-(4-(pyridin-3-yl)butyl)acrylamide

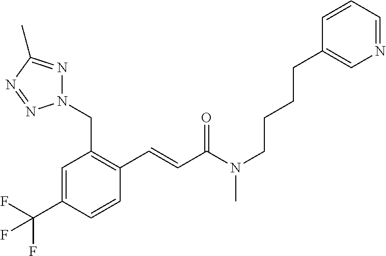 [SMILES: Cc1nnn(Cc2cc(C(F)(F)F)ccc2/C=C/C(=O)N(C)CCCCc2cccnc2)n1]

LCMS: Rt=2.72 mins; MS m/z 459.4 [M+H]+; Method 8minLowpHv01.

### Example 6.7

### (E)-N-Methyl-3-(2-((5-methyl-2H-tetrazol-2-yl)methyl)-4-(trifluoromethyl)phenyl)-N-((4-phenylthiazol-2-yl)methyl)acrylamide

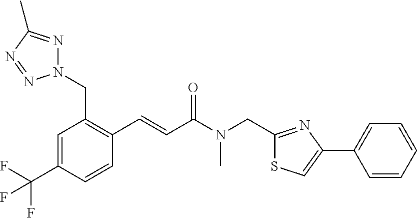 [SMILES: Cc1nnn(Cc2cc(C(F)(F)F)ccc2/C=C/C(=O)N(C)Cc2nc(-c3ccccc3)cs2)n1]

LCMS: Rt=1.46 mins; MS m/z 500.5 [M+H]+; Method 2minLowpHv03.

### Example 6.8

### (E)-N-((1-(tert-Butyl)-5-oxopyrrolidin-3-yl)methyl)-N-methyl-3-(2-((5-methyl-2H-tetrazol-2-yl)methyl)-4-(trifluoromethyl)phenyl)acrylamide

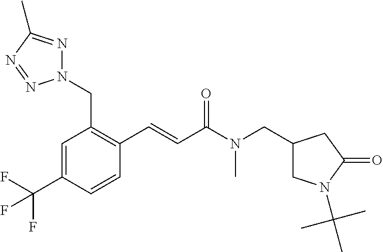 [SMILES: Cc1nnn(Cc2cc(C(F)(F)F)ccc2/C=C/C(=O)N(C)CC2CC(=O)N(C(C)(C)C)C2)n1]

LCMS: Rt=1.26 mins; MS m/z 479.4 [M+H]+; Method 2minLowpHv03.

### Example 6.9

### (E)-N-Methyl-3-(2-((5-methyl-2H-tetrazol-2-yl)methyl)-4-(trifluoromethyl)phenyl)-N-((3-phenyl-1,2,4-oxadiazol-5-yl)methyl)acrylamide

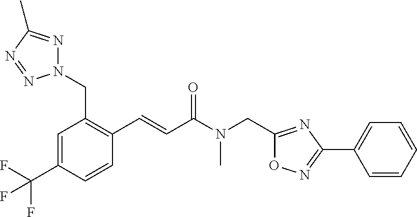 [SMILES: Cc1nnn(Cc2cc(C(F)(F)F)ccc2/C=C/C(=O)N(C)Cc2nc(-c3ccccc3)no2)n1]

LCMS: Rt=1.41 mins; MS m/z 484.2 [M+H]+; Method 2minLowpHv03.

### Example 6.10

### (E)-N-(Benzo[d]thiazol-2-ylmethyl)-N-methyl-3-(2-((5-methyl-2H-tetrazol-2-yl)methyl)-4-(trifluoromethyl)phenyl)acrylamide

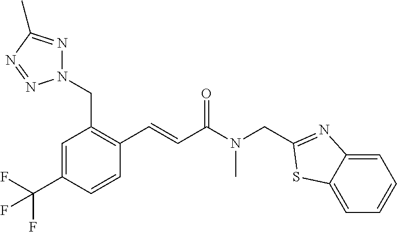 [SMILES: Cc1nnn(Cc2cc(C(F)(F)F)ccc2/C=C/C(=O)N(C)Cc2nc3ccccc3s2)n1]

LCMS: Rt=1.4 mins; MS m/z 473.3 [M+H]+; Method 2minLowpHv03.

### Preparation of Intermediates

### Intermediate A

### (E)-3-(4-Chloro-2-((5-methyl-2H-tetrazol-2-yl)methyl)phenyl)acrylic acid

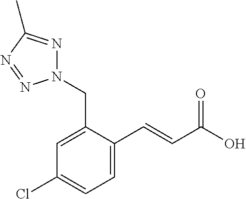 [SMILES: Cc1nnn(Cc2cc(Cl)ccc2/C=C/C(=O)O)n1]

### Step 1: 2-(2-Bromo-5-chlorobenzyl)-5-methyl-2H-tetrazole

5-Methyl-2H-tetrazole (77 g, 913 mmol) was placed in a flask with dry DMF (400 mL) at 0° C. using an ice bath. Potassium carbonate (168 g, 1217 mmol) was added portionwise followed by dropwise addition of 1-bromo-2-(bromomethyl)-4-chlorobenzene (173 g, 608 mmol) in DMF (400 mL) and the resulting mixture was stirred at room temperature for 2 h. The mixture was poured into water and the resulting suspension was collected by filtration. The solid was triturated with iso-hexane and the undissolved solid was removed by filtration. The filtrate was concentrated under reduced pressure giving a white solid which was suspended in water and stirred overnight. The product was filtered and washed with water to afford the title compound;

LCMS: Rt 1.15 mins; MS m/z 289.0 [M+H]+; 2minLowpHv01

### Step 2. (E)-Ethyl 3-(4-chloro-2-((5-methyl-2H-tetrazol-2-yl)methyl)phenyl)acrylate

2-(2-Bromo-5-chlorobenzyl)-5-methyl-2H-tetrazole (step 1) (15 g, 52.2 mmol), tri-o-tolylphosphine (0.794 g, 2.61 mmol) and triethylamine (10.56 g, 104 mmol) were placed in a flask with dry, degassed DMF (80 mL). Ethyl acrylate (7.83 g, 78 mmol) was added followed by palladium diacetate (0.586 g, 2.61 mmol) and the reaction mixture was stirred at 100° C. overnight. The mixture was allowed to cool and diluted with EtOAc (150 mL) and filtered to remove any precipitated palladium (also some insoluble salts). The reaction mixture was partitioned between EtOAc and water. The organic phase was washed with water and brine, dried over MgSO4, filtered and the solvent was removed in vacuo. When ˜75% of the solvent was removed, a solid precipitated out which was filtered collected by filtration and dried to afford the title compound as a white solid;

¹H NMR (400 MHz, DMSO-d6) δ 7.92 (1H, d), 7.89 (1H, d), 7.59 (1H, d), 7.51 (1H, d of d), 6.59 (1H, d), 6.09 (2H, s), 4.20 (2H, q), 2.41 (3H, s), 1.26 (3H, t),

### Step 3. (E)-3-(4-Chloro-2-((5-methyl-2H-tetrazol-2-yl)methyl)phenyl)acrylic acid

(E)-Ethyl 3-(4-chloro-2-((5-methyl-2H-tetrazol-2-yl)methyl)phenyl)acrylate (8.75 g, 28.5 mmol) was placed in a flask with EtOH (100 mL). 2M NaOH (57.1 mL, 114 mmol) was added and the reaction mixture was stirred at room temperature overnight. The ethanol was removed in vacuo and the reaction mixture was acidified with 2M HCl. The resulting precipitate was collected by filtration, washed with water and dried to afford the title compound as a white solid;

LCMS: Rt 0.99 mins; MS m/z 279.2 [M+H]+; Method 2minLowpHv01

### Intermediate AB

### (E)-3-(2-((5-Methyl-2H-tetrazol-2-yl)methyl)-4-(trifluoromethyl)phenyl)acrylic acid

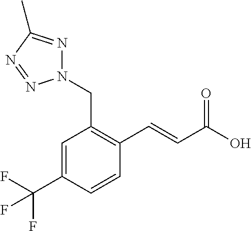 [SMILES: Cc1nnn(Cc2cc(C(F)(F)F)ccc2/C=C/C(=O)O)n1]

### Step 1: 2-(2-Bromo-5-(trifluoromethyl)benzyl)-5-methyl-2H-tetrazole

To a stirred solution of 5-methyl-2H-tetrazole (19.44 g, 231 mmol) in DMF (154 mL) at 10° C. under N₂ was added K₂CO₃ (42.6 g, 308 mmol). The resulting suspension was cooled to −2° C. (ice salt bath) and a solution of 1-bromo-2-(bromomethyl)-4-(trifluoromethyl)benzene (49 g, 154 mmol) in DMF (66 mL) was added dropwise over 30 mins keeping the internal T below 5° C. On complete addition, the mixture was allowed to warm to room temperature and the resulting white suspension stirred overnight. Water (400 mL) was added slowly to the mixture which was then extracted with EtOAc (2×500 mL). The combined organic extracts were washed with brine (500 mL), dried (MgSO4) and concentrated in vacuo to yield a colourless oil. On standing a white crystalline solid formed which was suspended in a colourless oil. Iso-hexane (150 mL) was added and the resulting slurry was filtered and washed with iso-hexane (2×50 mL). The filtrate was concentrated in vacuo to yield a colourless oil. Purification of the oil by chromatography on silica eluting with 0-50% EtOAc in iso-hexane afforded the title compound;

LCMS: Rt 1.30 mins; MS m/z 321.3 [M+H]+; Method 2minLowpHv03

### Step 2. (E)-Ethyl 3-(2-((5-methyl-2H-tetrazol-2-yl)methyl)-4-(trifluoromethyl)phenyl)acrylate

To a stirred solution of 2-(2-bromo-5-(trifluoromethyl)benzyl)-5-methyl-2H-tetrazole (step 1) (17 g, 52.9 mmol) in DMF (76 mL) was added tri-o-tolylphosphine (0.806 g, 2.65 mmol) and triethylamine (14.76 mL, 106 mmol). The solution was de-gassed by bubbling N₂ through for 20 mins. Pd(OAc)₂ (0.594 g, 2.65 mmol) and ethyl acrylate (8.66 mL, 79 mmol) were added and the reaction mixture heated to 90° C. under N₂. After cooling to room temperature, the mixture was partitioned between water (150 mL) and EtOAc (250 mL). The phases were separated and the aqueous phase extracted with more EtOAc (250 mL). The combined organic layers were washed with brine (2×250 mL), dried (MgSO4) and concentrated in vacuo to yield the title compound as an orange oil;

LCMS: Rt 1.36 mins; MS m/z 341.5 [M+H]+; Method 2minLowpHv03

### Step 3: (E)-3-(2-((5-Methyl-2H-tetrazol-2-yl)methyl)-4-(trifluoromethyl)phenyl)acrylic acid

To a stirred solution of crude (E)-ethyl 3-(2-((5-methyl-2H-tetrazol-2-yl)methyl)-4-(trifluoromethyl)phenyl)acrylate (step 2) (18.02 g, assume 53.0 mmol) in EtOH (212 mL) was added 2M NaOH (79 mL, 159 mmol) slowly. The resulting orange solution stirred at room temperature overnight. The resulting mixture was concentrated in vacuo to a volume of 100 ml and then filtered. 5M HCl (38 mL) was added slowly to adjust the pH to 2 whereupon a solid started to crystallise out of solution. The mixture was stirred at room temperature for 2 h to allow full crystallisation. The resulting slurry was filtered, washing the filter cake with 50% aq. EtOH (2×20 mL). The solid was dried in vacuo at 40° C. overnight to afford the title compound;

LCMS: Rt 1.14 mins; MS m/z 313.4[M+H]+; Method 2minLowpHv03

### Intermediate B

### (E)-N-Methyl-3-(2-((5-methyl-2H-tetrazol-2-yl)methyl)-4-(trifluoromethyl)phenyl)-N-(piperidin-4-ylmethyl)acrylamide hydrochloride

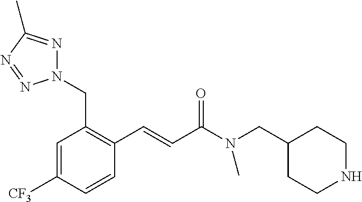 [SMILES: Cc1nnn(Cc2cc(C(F)(F)F)ccc2/C=C/C(=O)N(C)CC2CCNCC2)n1]

### Step 1: (E)-tert-butyl 4-((N-methyl-3-(2-((5-methyl-2H-tetrazol-2-yl)methyl)-4-(trifluoromethyl)phenyl)acrylamido)methyl)piperidine-1-carboxylate

To (E)-3-(2-((5-methyl-2H-tetrazol-2-yl)methyl)-4-(trifluoromethyl)phenyl)acrylic acid (Intermediate AB) (369 mg, 1.182 mmol) and tert-butyl 4-((methylamino)methyl)piperidine-1-carboxylate (270 mg, 1.182 mmol) in DCM (6 mL) was added triethylamine (0.824 mL, 5.91 mmol) followed by dropwise addition of T3P® (50% in DMF) (0.828 mL, 1.418 mmol) and the mixture was stirred at room temperature for 2 h.

The resulting product mixture was partitioned between EtOAc and water and the aqueous layer was removed. The organic layer was washed with saturated sodium bicarbonate solution, water, brine and dried using a phase separating column and concentrated in vacuo. Purification of the crude product by chromatography on silica eluting with 0-100% EtOAc in iso-hexane afford the title compound;

LCMS: Rt=1.46 mins; MS m/z 523.6 [M+H]+; Method 2minLowpHv03.

### Step 2. (E)-N-methyl-3-(2-((5-methyl-2H-tetrazol-2-yl)methyl)-4-(trifluoromethyl)phenyl)-N-(piperidin-4-ylmethyl)acrylamide hydrochloride

To (E)-tert-butyl 4-((N-methyl-3-(2-((5-methyl-2H-tetrazol-2-yl)methyl)-4-(trifluoromethyl)phenyl)acrylamido)methyl)piperidine-1-carboxylate (step 1) (1.4 g, 2.68 mmol) in DCM (12 ml) was added TFA (2.477 ml, 32.1 mmol) and the mixture was stirred at room temperature for 4 h. The solvent was removed under reduced pressure and the crude product was loaded onto a pre-wetted (MeOH) Isolute® SCX-2 cartridge eluting with MeOH followed by 2M NH₃ in MeOH. The methanolic ammonia fractions were concentrated under reduced pressure and the resulting film was treated with diethyl ether giving a hazy solution. HCl (2M in diethyl ether) (2.68 ml, 5.36 mmol) was added and the mixture was stirred. The resulting white precipitate was collected by filtration to afford the title compound;

LCMS: Rt=0.80 mins; MS m/z 424.4 [M+H]+; Method 2minLowpHv03.

### Biological Data:

The compounds of the invention are suitable as ATX inhibitors and may be tested in the following assays.

Reagents—LPC (oleoyl (18:1)) was purchased from Avanti Polar Lipids (Alabaster, Ala.) and solubilized in methanol to 20 mM. Amplex Red was obtained from Invitrogen Life Technologies (Paisley, UK) and dissolved in DMSO to 10 mM. Choline oxidase and horseradish peroxidase (HRP) were obtained from Sigma Aldrich (Dorset, UK) and dissolved in HBSS to 20 U/ml and 200 U/ml respectively. All reagents were stored at −20° C. in single use aliquots. All experimental measurements were performed in assay buffer made up immediately prior to use (HBSS, 0.01% BSA essentially fatty acid free).

Protein—Recombinant human ATX was prepared at Novartis (Basel, CH) in a human embryonic kidney (HEK) cell preparation, and stored in single use aliquots of 26 mg/ml (26 μM) stocks stored at −80° C.

Method—All experimental measurements were performed in black 384 well polystyrene (low volume, round bottom, Corning (3676)) plates. PerkinElmer EnVision (Fluorescence Intensity/Absorbance Monochromator) or Tecan Infinite 200 PRO series plate reader was used to detect change in fluorescent intensity.

Assessing ATX inhibition—ATX activity was determined by measurement of released choline in reactions containing ATX (10 nM), choline oxidase (0.1 U/ml), HRP (100 U/ml), amplex red (50 μM) and LPC 18:1 (10 μM). Compounds of the invention were prepared as 10 point serial dilutions from 1 μM in duplicate and pre-incubated with ATX at 37° C. for 20 minutes prior to the addition of remaining reagents. The liberated choline was measured from changes in fluorescence intensity (λex 530 nm, λem 590 nm) of the product resurofin at 37° C. every 2 minutes over a 40-minute period. ATX activity was measured as a slope of the linear portion of the progress curve, typically between 14 to 24 minutes.

Data analysis—Slope data was exported to Graphpad prism (Graphpad software, San Diego, Calif.) where data was fitted to equation 1.

Y=Bottom+(Top−Bottom)/(1+10̂((LogIC50−X)*HillSlope)) Equation 1:

IC₅₀ values are determined from the concentration of compound that reduced the total activity by 50% and represent the mean of n≧2.

Table 1: The following table gives the IC₅₀ values for the exemplified compounds as measured in the above assay.

TABLE 1

| Example no. | IC₅₀ (μM) |
|---|---|
| 1.1 | 0.014 |
| 1.2 | 0.02 |
| 2.1 | 0.064 |
| 2.2 | 0.074 |
| 2.3 | 0.073 |
| 2.4 | 0.05 |
| 2.5 | 0.094 |
| 2.6 | 0.052 |
| 2.7 | 0.183 |
| 3 | 0.01 |
| 4.1 | 0.0049 |
| 4.2 | 0.0045 |
| 5 | 0.013 |
| 6.1 | 0.0069 |
| 6.2 | 0.009 |
| 6.3 | 0.04 |
| 6.4 | 0.013 |
| 6.5 | 0.016 |
| 6.6 | 0.049 |
| 6.7 | 0.006 |
| 6.8 | 0.007 |
| 6.9 | 0.0053 |
| 6.10 | 0.012 |

1. A compound of formula (I) 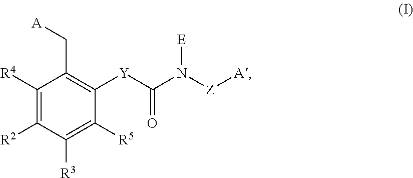
or a pharmaceutically acceptable salt thereof,
wherein
A is selected from the group consisting of 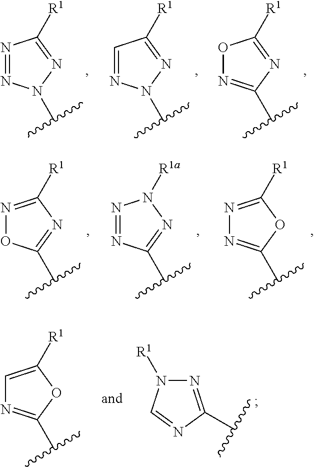
R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) 5 or 6 membered heteroaryl which heteroaryl is unsubstituted or substituted with 1 to 3 substituents independently selected from the group consisting of X;
(ii) phenyl which phenyl is unsubstituted or substituted with 1 to 3 substituents independently selected from the group consisting of X;
(iii) 5 to 10 membered fused bicyclic ring system which 5 to 10 membered fused bicyclic ring system is unsubstituted or substituted with 1 to 3 substituents independently selected from the group consisting of X;
(iv) 5 or 6 membered heterocyclyl which heterocyclyl is unsubstituted or substituted with 1 to 3 substituents independently selected from the group consisting of X;
(v) C₁₋₄ alkyl;
(vi) C₁₋₄ alkoxy;
(vii) C₁₋₄ alkoxy C₁₋₄ alkyl;
(viii) C₁₋₄ haloalkyl;
(ix) hydroxy C₁₋₄ alkyl;
(x) C₃₋₆ cycloalkyl;
(xi) —C(═O)R^{Bc};
(xii) —C(═O)OR^{Be};
(xiii) —NR^{Bd}—C(═O)R^{Bc};
(xiv) —NR^{Bd}—C(═O)OR^{Bc};
(xv) —C(═O)NR^{Bc}R^{Bd};
(xvi) —NR^{Bd}R^{Be};
(xvii) —NR^{Bd}—S(O)₂—R^{Bf};
(xviii) —S(O)₂—NR^{Bd}R^{Be};
(xix) —S(O)₂—R^{Bf};
(xx) halogen;
(xxi) OH;
(xxii) oxo; and
(xxiii) CN;
R^{Bc}, R^{Be} and R^{Bf} are independently selected from the group consisting of H, C₁₋₄alkyl, —(CR^{Ba}R^{Bb})ₙ—C₃₋₆cycloalkyl, hydroxyC₁₋₄alkyl, C₁₋₄haloalkyl, C₁₋₄alkoxyC₁₋₄alkyl, OH, —(CR^{Ba}R^{Bb})ₙ-5 or 6 membered heteroaryl, —(CR^{Ba}R^{Bb})ₙ-phenyl and —(CR^{Ba}R^{Bb})ₙ-5 or 6 membered heterocyclyl, wherein the C₃₋₆cycloalkyl, heteroaryl, phenyl and heterocyclyl are unsubstituted or substituted by 1 to 3 substituents selected from the group consisting of X;
R^{Bd} is selected from the group consisting of H and C₁₋₄alkyl; or
R^{Bd} and R^{Be} together with the nitrogen atom to which they are attached form a 5 to 6 membered heterocyclyl which heterocyclyl is unsubstituted or substituted by 1 to 3 substituents selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆ cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Ba}, R^{Bb}, R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
n and q are independently selected from the group consisting of 0, 1, 2, 3 and 4.

2-3. (canceled)

4. A compound according to claim 1, of formula (I) 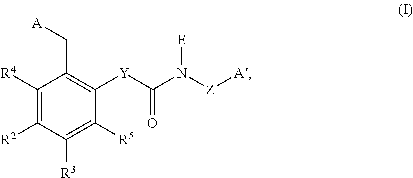
or a pharmaceutically acceptable salt thereof,
wherein
A is selected from the group consisting of 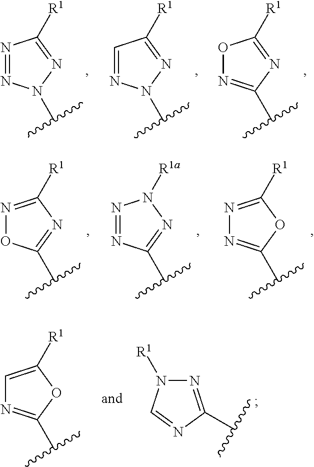
R¹ is selected from the group consisting of H and C₁₋₄ alkyl;
R^{1a} is C₁₋₄ alkyl;
R² is halogen, —CF₃, —CF₂H, —OCF₃, —OCF₂H, —OCH₃, —CH₃ or CN, and R³, R⁴ and R⁵ are H; or
R³ is halogen and R², R⁴ and R⁵ are H; or
R⁴ is halogen and R², R³ and R⁵ are H; or
R² is halogen, —CF₃, —CF₂H, —OCF₃, —OCF₂H, —OCH₃, —CH₃ or CN, R³ is halogen and R⁴ and R⁵ are H;
Y is selected from the group consisting of —CH═CH—, —CH₂—CH₂—, —O—CH₂—, —CH₂—O—, —C(CH₃)═CH— and —C═C(CH₃)—;
E is selected from the group consisting of H and C₁₋₆ alkyl;
Z is selected from the group consisting of —(CR^{7a}R^{7b})ₘ— and —(CR^{7a}R^{7b})ₘ—O—;
R^{7a} and R^{7b} is independently selected from H, OH, C₁₋₄ alkyl and C₁₋₄ alkoxy;
m is selected from the group consisting of 0, 1, 2, 3 and 4;
A′ is selected from the group consisting of
(i) -piperidin-4-yl;
(ii) -pyrrolidin-3-yl;
(iii) -oxadiazol-5-yl;
(iv) -thiazol-2-yl;
(v) -pyrazol-5-yl;
(vi) -pyrazol-3-yl;
(vii) -isoxazol-5-yl;
(viii) -tetrahydrofuran-2-yl
each of (i) to (vii) is unsubstituted or substituted with 1 to 3 substituents independently selected from the group consisting of X;
X is independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, —(CR^{Xa}R^{Xb})_{q}—C₃₋₆cycloalkyl, halogen, CN, OH, oxo, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl, —(CR^{Xa}R^{Xb})_{q}—C(═O)R^{Xc}, —(CR^{Xa}R^{Xb})_{q}—C(═O)OR^{Xc}, —(CR^{Xa}R^{Xb})_{q}—NR^{Xd}R^{Xe}, —(CR^{Xa}R^{Xb})_{q}—C(═O)NR^{Xd}R^{Xe}, —(CR^{Xa}R^{Xb})_{q}—NR^{Xd}—C(═O)R^{Xe} and —(CR^{Xa}R^{Xb})_{q}—NR^{Xd}—S(O)₂—R^{Xf}, —(CR^{Xa}R^{Xb})_{q}—S(O)₂—NR^{Xd}R^{Xe} and —(CR^{Xa}R^{Xb})_{q}—S(O)₂—R^{Xf}; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH;
R^{Xa} and R^{Xb} are independently selected from the group consisting of H, OH, C₁₋₄ alkyl and C₁₋₄ alkoxy;
R^{Xc}, R^{Xd}, R^{Xe} and R^{Xf} are independently selected from the group consisting of H, C₁₋₄alkyl, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl, OH, —(CR^{Xa}R^{Xb})_{q}-5 or 6 membered heterocyclyl, —(CR^{Xa}R^{Xb})_{q}-5 or 6 membered heteroaryl, —(CR^{Xa}R^{Xb})_{q}-phenyl; wherein the C₃₋₆cycloalkyl, heteroaryl, phenyl and heterocyclyl are unsubstituted or substituted by 1 to 3 substituents independently selected from the group consisting of C₁₋₄ alkyl, C₁₋₄ alkoxy, C₁₋₄ haloalkyl, hydroxyC₁₋₄alkyl, C₁₋₄alkoxyC₁₋₄alkyl, halogen and OH; or
R^{Xd} and R^{Xe} together with the nitrogen atom to which they are attached form a 5 to 6 membered heterocyclyl which heterocyclyl is unsubstituted or substituted by 1 to 3 substituents selected from the group consisting of H, C₁₋₄alkyl, C₁₋₄alkoxy, —(CR^{Xa}R^{Xb})_{q}—C₃₋₆cycloalkyl, hydroxyC₁₋₄alkyl, C₁₋₄haloalkyl, C₁₋₄alkoxyC₁₋₄alkyl and OH;
q is selected from the group consisting of 0, 1 and 2.

5. A compound or pharmaceutically acceptable salt thereof according to claim 1, wherein
R² is halogen, —CF₃, —CF₂H, —OCF₃, —OCF₂H, —OCH₃, —CH₃ or CN, and R³, R⁴ and R⁵ are H.

6. A compound or pharmaceutically acceptable salt thereof according to claim 1, wherein A is selected from the group consisting of

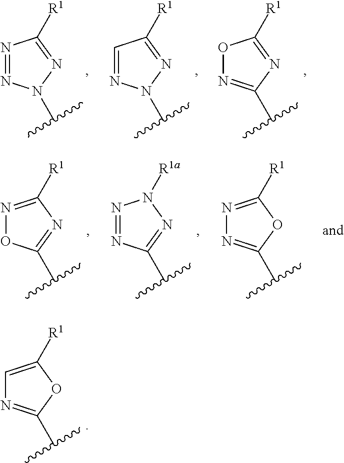

7. A compound according to claim 1 selected from the group consisting of,
(E)-tert-butyl 4-((3-(4-chloro-2-((5-methyl-2H-tetrazol-2-yl)methyl)phenyl)-N-methylacrylamido)methyl)piperidine-1-carboxylate;
(E)-tert-butyl 4-(N-ethyl-3-(2-((5-methyl-2H-tetrazol-2-yl)methyl)-4-(trifluoromethyl)phen l)acrylamido)piperidine-1-carboxylate;
(E)-3-(4-chloro-2-((5-methyl-2H-tetrazol-2-yl)methyl)phenyl)-N-ethyl-N-(pyridin-4-ylmethyl)acrylamide;
(E)-3-(4-chloro-2-((5-methyl-2H-tetrazol-2-yl)methyl)phenyl)-N-methyl-N-((4-methylthiazol-2-yl)methyl)acrylamide;
(E)-3-(4-chloro-2-((5-methyl-2H-tetrazol-2-yl)methyl)phenyl)-N-methyl-N-((3-methylisoxazol-5-yl)methyl)acrylamide;
(E)-3-(4-chloro-2-((5-methyl-2H-tetrazol-2-yl)methyl)phenyl)-N-ethyl-N-(2-methoxyethyl)acrylamide;
(E)-3-(4-chloro-2-((5-methyl-2H-tetrazol-2-yl)methyl)phenyl)-N-ethyl-N-(2-hydroxy-2-(3-hydroxyphenyl)ethyl)acrylamide;
(E)-N-((1-tert-butyl-5-oxopyrrolidin-3-yl)methyl)-3-(4-chloro-2-((5-methyl-2H-tetrazol-2-yl)methyl)phenyl)-N-methylacrylamide;
(E)-3-(4-chloro-2-((5-methyl-2H-tetrazol-2-yl)methyl)phenyl)-N-methyl-N-((tetrahydrofuran-2-yl)methyl)acrylamide;
(E)-N-((1-(4-fluorobenzoyl)piperidin-4-yl)methyl)-N-methyl-3-(2-((5-methyl-2H-tetrazol-2-yl)methyl)-4-(trifluoromethyl)phenyl)acrylamide;
(E)-N-((1-(cyclopropylsulfonyl)piperidin-4-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-((1-tosylpiperidin-4-yl)methyl)acrylamide;
(E)-N-((1-(4-fluorobenzyl)piperidin-4-yl)methyl)-N-methyl-3-(2-((5-methyl-2H-tetrazol-2-yl)methyl)-4-(trifluoromethyl)phenyl)acrylamide;
(E)-N-((1,3-dimethyl-H-pyrazol-5-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-(2-phenoxyethyl)acrylamide;
(E)-N-methyl-3-(2-((5-methyl-2H-tetrazol-2-yl)methyl)-4-(trifluoromethyl)phenyl)-N-((4-methylthiazol-2-yl)methyl)acrylamide;
(E)-N-methyl-3-(2-((5-methyl-2H-tetrazol-2-yl)methyl)-4-(trifluoromethyl)phenyl)-N-((5-phenyl-1,3,4-oxadiazol-2-yl)methyl)acrylamide;
(E)-N-(2-(4-methoxybenzylamino)-2-oxoethyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-(4-(pyridin-3-yl)butyl)acrylamide;
(E)-N-methyl-3-(2-((5-methyl-2H-tetrazol-2-yl)methyl)-4-(trifluoromethyl)phenyl)-N-((4-phenylthiazol-2-yl)methyl)acrylamide;
(E)-N-((1-tert-butyl-5-oxopyrrolidin-3-yl)methyl)-N-methyl-3-(2-((5-methyl-2H-tetrazol-2-yl)methyl)-4-(trifluoromethyl)phenyl)acrylamide;
(E)-N-methyl-3-(2-((5-methyl-2H-tetrazol-2-yl)methyl)-4-(trifluoromethyl)phenyl)-N-((3-phenyl-1,2,4-oxadiazol-5-yl)methyl)acrylamide; and
(E)-N-(benzo[d]thiazol-2-ylmethyl)-N-methyl-3-(2-((5-methyl-2H-tetrazol-2-yl)methyl)-4-(trifluoromethyl)phenyl)acrylamide;
or a pharmaceutically acceptable salt thereof.

8. A pharmaceutical composition comprising a compound according to claim 1, or a pharmaceutically acceptable salt thereof, and a pharmaceutically acceptable carrier.

9. A pharmaceutical combination comprising a therapeutically effective amount of the compound according to claim 1, or a pharmaceutically acceptable salt thereof, and one or more therapeutically active co-agent.

10-13. (canceled)

14. A method of treating a disease or condition selected from fibrosis, pruritus, cirrhosis, cancer, diabetes, kidney diseases and pain comprising administering to the subject a therapeutically effective amount of a compound according to claim 1.

15. (canceled)

16. A method of treating an autotaxin dependent or an autotaxin mediated disease or condition comprising administering to a subject a therapeutically effective amount of a compound according to claim 1.

17. The method according to claim 14, wherein the disease or condition is idiopathic pulmonary fibrosis.
